(19) United States
(12) Patent Application Publication
(10) Pub. No.: US 2009/0253778 A1
(43) Pub. Date: Oct. 8, 2009

(54) DNA composition against tumor stromal antigen FAP and methods of use thereof

(76) Inventors: Ralph A. Reisfeld, La Jolla, CA (US); Markus Loeffler, Avon (FR); Jorg A. Kruger, Berlin (DE); Andreas G. Niethammer, La Jolla, CA (US)

Correspondence Address: Olson & Cepuritis, LTD., 20 NORTH WACKER DRIVE, 36TH FLOOR, CHICAGO, IL, 60606, US

(21) Appl. No.: 11/820,823

(22) Filed: Jun. 21, 2007

Related U.S. Application Data
(60) Provisional application No. 60/815,316, filed on Jun. 21, 2006.

(51) Int. Cl. A61K 31/7088; C12N 15/74; A61P 35/00

(52) U.S. Cl. 514/44 R; 435/320.1

(57) ABSTRACT

A DNA composition effective for eliciting an immune response against tumor cells and tumor metastases comprising a DNA construct that encodes for at least one epitope of fibroblast activation protein (FAP), which is expressible in immune cells, and which is incorporated in a pharmaceutically acceptable carrier. The composition can encode a single epitope of FAP, a polypeptide comprising two or more epitopes of FAP, the entire FAP protein, or any portion thereof that will elicit the desired immune response. In one preferred embodiment, the composition also includes a DNA construct that encodes an immune effector protein, such as a cytokine. The DNA composition of the invention can be used alone or in combination with a chemotherapeutic agent to treat diseases such as tumors and tumor metastases.

### CROSS-REFERENCE TO RELATED APPLICATIONS

This application claims the benefit of U.S. Provisional Application for Patent Ser. No. 60/815,316, filed on Jun. 21, 2006, which is incorporated herein by reference.

### GOVERNMENTAL RIGHTS

This invention was made with United States government support under Grant No. CA83856 from the National Institutes of Health and Grant No. BC031079 from the Department of Defense. The government has certain rights in this invention.

### FIELD OF THE INVENTION

This invention relates to deoxyribonucleic acid (DNA) compositions encoding suitable molecules effective for eliciting an immune response against stromal fibroblast cells in tumors. More particularly this invention relates to DNA compositions encoding for the stromal antigen known as fibroblast activation protein (FAP). This invention also relates to methods of using the DNA composition to inhibit tumor growth and tumor metastases and to enhance cellular uptake of chemotherapeutic agents.

### BACKGROUND OF THE INVENTION

It has become increasingly clear that tumor antigens are elusive targets for cancer therapy. This can be explained by a variety of characteristics unique to tumor cells, including mutations of their antigens and inadequate antigen presentation due to down-regulation of MHC-antigen expression. Moreover, defects in apoptosis signaling pathways, as well as up-regulation of apoptosis-inhibitors such as survivin, XIAP or anti-apoptotic members of the bcl-2 family not only confer resistance to T cell mediated killing, but also to the apoptosis-inducing effect of chemotherapeutic agents.

There is a growing recognition that the stromal compartment plays a crucial role in tumorigenesis and tumor invasion. Stromal cells have been shown to stimulate the transformation of normal epithelial cells, and to interchange growth factors, cytokines and chemoattractants to activate the adjacent extracellular matrix and to induce the selection and expansion of neoplastic cells. In one study, tumor cells injected in mice as a suspension reportedly were not tumorigenic, whereas injection of fragments of solid tumors containing their stroma reportedly led to tumor growth (see Singh et al. J. Exp. Med. 1992; 175:139-146). The activated extracellular matrix apparently confers chemoresistance mediated by β1 integrins, which adhere to fibronectin, leading to the activation of β1 integrin-stimulated tyrosine kinase, which in turn, suppresses chemotherapy-induced apoptosis. This phenomenon has been reported in doxorubicin-sensitive myeloma cells, which developed resistance after adherence to fibronectin. Recent reports indicate that tumor rejection can be achieved by modulating tumor stromal fibroblasts or by distributing the tumor stromal network. In addition, tumor-associated macrophages and fibroblasts reportedly contribute to the local immuno-suppressive environment due to their ability to synthesize proteins, such as VEGF, TGF-β1 and IL-10. VEGF functions as an inhibitor of dendritic cell maturation and thus leads to tolerant T cells. VEGF is also a major factor involved in tumor-angiogenesis. Activation of VEGF leads to upregulation of survivin and many other apoptosis-inhibiting proteins in tumor-endothelial cells, shielding them from the apoptotic effects of chemotherapy. In contrast, inhibition of TGF-β1 can result in tumor eradication and protection against metastasis. IL-10 inhibits the maturation of dendritic cells, and thereby suppresses the cell-mediated antitumor responses mediated by Th1 cells. This effect shifts the dendritic cells towards largely ineffective humoral immune responses.

Fibroblasts are connective tissue cells which secrete an extracellular matrix rich in collagen and other macromolecules. Fibroblasts in the tumor-stroma (i.e., tumor supporting tissue) synthesize fibroblast activation protein (FAP), a type II transmembrane protein that functions as a serine protease. FAP is selectively overexpressed in over 90% of stromal fibroblasts associated with colon, breast and lung carcinomas. Transient overexpression of FAP can also be observed during wound healing and in some fetal mesenchymal tissues. Additionally, this enzyme reportedly cleaves gelatin and type I collagen, and has been implicated in extracellular matrix remodeling. Overexpression of FAP reportedly leads to promotion of tumor growth and increases in metastatic potential, whereas treatment with anti-FAP antibodies inhibits tumor growth. Furthermore, tumor-stromal expression of collagen type I, which is mainly produced by fibroblasts, inversely correlates with intratumor uptake of various compounds, including chemotherapeutic agents, thereby sustaining the involvement of the stromal compartment in chemotherapy resistance.

Vaccines have been utilized to provide a long term protection against a number of disease conditions by very limited administration of a prophylactic agent that stimulates an organism's immune system to destroy disease pathogens before they can proliferate and cause a pathological effect. Various approaches to vaccines and vaccinations are described in Bernard R. Glick and Jack J. Pasternak, Molecular Biotechnology, Principles and Applications of Recombinant DNA, Second Edition, ASM Press pp. 253-276 (1998).

Vaccination is a means of inducing the body's own immune system to seek out and destroy an infecting agent before it causes a pathological response. Typically, vaccines are either live, but attenuated, infectious agents (virus or bacteria), or a killed form of the agent. A vaccine consisting of a live bacteria or virus must be non-pathogenic. Typically, a bacterial or viral culture is attenuated (weakened) by physical or chemical treatment. Although the agent is nonvirulent, it can still elicit an immune response in a subject treated with the vaccine.

An immune response is elicited by antigens, which can be either specific macromolecules or an infectious agent. These antigens are generally either proteins, polysaccharides, lipids, or glycolipids, which are recognized as “foreign” by lymphocytes known as B cells and T cells. Exposure of both types of lymphocytes to an antigen elicits a rapid cell division and differentiation response, resulting in the formation of clones of the exposed lymphocytes. B cells produce plasma cells, which in turn, produce proteins called antibodies (Ab), which selectively bind to the antigens present on the infectious agent, thus neutralizing or inactivating the pathogen (humoral immunity). In some cases, B cell response requires the assistance of CD4 helper T cells.

The specialized T cell clone that forms in response to the antigen exposure is a cytotoxic T lymphocyte (CTL), which is capable of binding to and eliminating pathogens and tissues that present the antigen (cell-mediated or cellular immunity). In some cases, an antigen presenting cell (APC) such as a dendritic cell, will envelop a pathogen or other foreign cell by endocytosis. The APC then processes the antigens from the cells and presents these antigens in the form of a histocompatibility molecule: peptide complex to the T cell receptor (TCR) on CTLs, thus stimulating an immune response.

Humoral immunity, characterized by the formation of specific antibodies, is generally most effective against acute bacterial infections and repeat infections from viruses, whereas cell-mediated immunity is most effective against viral infection, chronic intracellular bacterial infection, and fungal infection. Cellular immunity is also known to protect against certain cancers and is responsible for rejection of organ transplants.

Antibodies to antigens from prior infections remain detectable in the blood for very long periods of time, thus affording a means of determining prior exposure to a pathogen. Upon re-exposure to the same pathogen, the immune system effectively prevents reinfection by eliminating the pathogenic agent before it can proliferate and produce a pathogenic response.

The same immune response that would be elicited by a pathogen can also sometimes be produced by a non-pathogenic agent that presents the same antigen as the pathogen. In this manner, the subject can be protected against subsequent exposure to the pathogen without having previously fought off an infection.

Not all infectious agents can be readily cultured and inactivated, as is required for vaccine formation, however. Modern recombinant DNA techniques have allowed the engineering of new vaccines to seek to overcome this limitation. Infectious agents can be created that lack the pathogenic genes, thus allowing a live, nonvirulent form of the organism to be used as a vaccine. It is also possible to engineer a relatively nonpathogenic organism such as E. coli to present the cell surface antigens of a pathogenic carrier. The immune system of a subject vaccinated with such a transformed-carrier is “tricked” into forming antibodies to the pathogen. The antigenic proteins of a pathogenic agent can be engineered and expressed in a nonpathogenic species and the antigenic proteins can be isolated and purified to produce a “subunit vaccine.” Subunit vaccines have the advantage of being stable, safe, and chemically well defined; however, their production can be cost prohibitive.

A new approach to vaccines has emerged in recent years, broadly termed genetic immunization. In this approach, a nucleic acid (polynucleotide) encoding an antigen of a pathogenic agent is operably inserted into cells in the subject to be immunized. The treated cells, preferably antigen presenting cells (APCs) such as the dendritic cells, are transformed and produce the antigenic proteins of the pathogen. These in vivo-produced antigens then trigger the desired immune response in the host. The genetic material utilized in such genetic vaccines can be either a DNA or RNA construct. Often the polynucleotide encoding the antigen is introduced in combination with other promoter polynucleotide sequences to enhance insertion, replication, or expression of the gene.

DNA vaccines encoding antigen genes can be introduced into the host cells of the subject by a variety of delivery systems. These delivery systems include prokaryotic and viral delivery systems. For example, one approach is to utilize a viral vector, such as vaccinia virus incorporating the new genetic material, to innoculate the host cells. Alternatively, the genetic material can be incorporated in a plasmid vector or can be delivered directly to the host cells as a “naked” polynucleotide, i.e. simply as purified DNA. In addition, the DNA can be stably transfected into attenuated bacteria such as Salmonella typhimurium. When a patient is orally vaccinated with the transformed Salmonella, the bacteria are transported to Peyer's patches in the gut (i.e., secondary lymphoid tissues), which then stimulate an immune response.

DNA vaccines provide an opportunity to immunize against disease states that are not caused by traditional pathogens, such as genetic diseases and cancer. Typically, a genetic cancer vaccine introduces into APCs a gene that encodes an antigen, and the so transformed APCs produce antigens to a specific type of tumor cell. An effective general vaccine against a number of cancer types can thus entail numerous individual vaccines for each type of cancer cell to be immunized against.

There is a continuing need for a generally effective DNA composition, such as a vaccine, for immunization against various forms of cancer. The present invention satisfies this need.

### SUMMARY OF THE INVENTION

A DNA composition effective for inhibiting tumor growth and tumor metastases comprises a DNA construct that encodes at least one epitope of fibroblast activation protein (FAP), which is expressible in immune cells of a subject to which the DNA construct has been administered. The DNA construct is incorporated in a pharmaceutically acceptable carrier. The composition can encode a single epitope of FAP, a polypeptide comprising two or more epitopes of FAP, the entire FAP protein, or any portion thereof that will elicit the desired immune response against tumor stromal cells. Preferably, the DNA construct encodes for human FAP having the amino acid residue sequence consisting of SEQ ID NO: 2 or a protein that has at least 80% sequence similarity to SEQ ID NO: 2 and which includes at least one epitope of FAP.

The DNA construct can be a naked DNA construct, preferably in the form of a plasmid. Such naked DNA constructs can be incorporated in a liposome delivery vehicle, a polymeric delivery vehicle, electroporation, gene gun, and the like, if desired. In some preferred embodiments the DNA construct is incorporated in an attenuated viral vector or an attenuated bacterial vector.

In a preferred embodiment, the DNA construct is incorporated in an attenuated bacterial vector, such as an attenuated Salmonella typhimurium, most preferably the doubly attenuated (AroA⁻, dam⁻) strain of Salmonella typhimurium.

Optionally, the DNA composition can also comprise a DNA construct encoding an immune effector protein, such as a cytokine. Preferred cytokines include CCL21, IL-2, and CD40LT.

The DNA compositions of the present invention can act as vaccines that target the tumor stromal fibroblast antigen FAP, which serves as a target for T cell-mediated cancer immunotherapy. Stromal fibroblasts are non-transformed cells present in the tumor environment, which support tumor growth. The approach of targeting FAP expressed by stromal fibroblast cells has several advantages over therapies directed against antigens that are solely expressed by tumor cells. First, stromal fibroblasts are genetically more stable than frequently mutating heterogeneous tumor cell populations. Accordingly, the expression of the target antigen remains more stable and serves as a more reliable target for immunotherapy. Second, antigen presentation from stromal fibroblasts to the T cell receptor complex are not impaired by downregulated MHC-antigen expression, as is frequently the case in tumor cells. Third, tumor cells often become increasingly resistant to T cell mediated killing due to defects in apoptosis signaling pathways, upregulation of antiapoptotic proteins, or immunosuppressive effects on CTLs. Additionally, targeting FAP, which is specifically overexpressed in the stromal compartment in over 90% of colon, breast and lung carcinomas allows for a therapeutic composition to treat a number of different malignancies, in contrast to therapies involving antigens that are expressed solely by specific tumor types.

In one preferred embodiment, the DNA compositions of the present invention break peripheral T cell tolerance against the FAP self-antigen by delivering its cDNA as an oral DNA composition with an attenuated bacterial delivery vector (e.g., an attenuated strain of Salmonella typhimurium) to antigen presenting cells in a secondary lymphoid organ, i.e., the Peyer's patches of the small intestine. In a prophylactic approach, the T cell-mediated antitumor immune response induced by vaccination with a DNA composition of the invention inhibited tumor growth in two different murine tumor models, i.e., in multi-drug resistant CT26 colon carcinoma, and multi-drug resistant D2F2 breast carcinoma. The present DNA compositions also significantly suppress the dissemination of established pulmonary metastases in a therapeutic model of CT26 colon carcinoma.

A preferred DNA composition comprises an attenuated Salmonella carrier, which is the doubly attenuated strain of S. typhimurium designated as RE 88 and which includes the dam⁻ and AroA⁻ mutations, available from Remedyne Corporation (Santa Barbara, Calif.). The attenuated Salmonella carrier includes DNA encoding at least one epitope of FAP, which is expressible in immune cells. The bacteria does not itself express FAP, but rather delivers the FAP DNA to immune cells (e.g., macrophages and dendritic cells), which in turn express FAP. Such compositions can prolong antitumor effects up to 10 months, and also achieve marked upregulation of T cell, NK cell and DC activation markers in murine models. Furthermore, in vivo depletion experiments of T cells indicated the only the involvement of CD8⁺ T cells, and no involvement of CD4⁺ T cells. The in vitro cytotoxic effect mediated by CD8⁺ T cells was specifically directed against target cells that overexpress the FAP antigen. For example, in a proof of concept experiment, CD8⁺ T cells from mice vaccinated with a composition of the invention induced in vitro cytotoxic lysis of tumor cells engineered to overexpress FAP, which indicates a specific immune response to FAP. Surprisingly, even though FAP can also be found transiently overexpressed during would healing, no impairment of wound healing caused by vaccination with the DNA compositions of the invention was observed.

The DNA compositions of the present invention also can incorporate DNA constructs that encode immune effector molecules as adjuvants for the composition. Such immune effector molecules include, for example, IL-2, an inducer of T cell proliferation; CCL21, a chemokine that chemo-attracts mature dendritic cells, and naive T cells; and CD40LT, a known inducer of dendritic cell maturation. The nucleic acids encoding the immune effector protein preferably are incorporated into a plasmid. The FAP and immune effector protein DNA constructs can be incorporated into the same plasmid or into two separate plasmids. The CTL-response induced against tumor-stromal fibroblasts can inhibit the growth of a variety of tumors, and is not specific to a particular tumor type.

The present invention also provides a method of inhibiting tumor growth and tumor metastases in a mammal comprising administering to a mammal a DNA composition of the invention in an amount sufficient to elicit an immune response against tumor-stromal cells that present the FAP antigen.

Another aspect of the present invention is an effective combination therapeutic regimen that involves administering a chemotherapeutic agent in conjunction with a DNA composition of the invention. In this method embodiment of the present invention various chemotherapeutic agents, such as doxorubicin, paclitaxel, and/or cyclophosphamide, which do not cause bone marrow suppression when administered at the maximum tolerable dose (MTD), are administered to the patient in conjunction with a DNA composition of the invention comprising a DNA construct that encodes FAP or an immunogenic portion thereof. The combination of a FAP DNA composition of the invention with such drugs offers an additional advantage, which is downregulation of collagen type I expression, and consequently, increased intratumoral uptake of various chemotherapeutic agents and other compounds. The DNA compositions of the present invention lead to increased tumor cell uptake of a variety of molecules, including, but not limited to antitumor chemotherapeutic agents. For example, the low-molecular weight dye fluorescein (376 Da), the high-molecular weight compound Evans blue albumin (68,500 Da), and the antitumor chemotherapeutic agent 5-fluorouracil (5-FU; 130 Da), all were taken up by tumor cells at much greater levels in mice treated with a DNA composition of the invention relative to mice treated with an inactive control composition. The combination of a DNA composition of the invention plus the antitumor drug doxorubicin induced complete rejection of orthotopically grown breast tumors in 50% of mice treated with the present combination therapy. Complete rejection of tumors was not observed in mice treated with a DNA composition alone or doxorubicin alone.

A preferred method provides a means for enhancing the uptake of a chemotherapeutic agent in a mammal. The method comprises administering to a mammal a DNA composition of the invention in an amount sufficient to elicit an immune response against cells in the mammal that present the FAP antigen and then administering to the mammal an effective amount of a chemotherapeutic agent.

Another preferred method embodiment is a method of inhibiting tumor growth or tumor metastases in a mammal comprising the steps of administering to a mammal a DNA composition of the invention in an amount sufficient to elicit an immune response against cells in the mammal that present the FAP antigen, and subsequently administering to the mammal an antitumor effective amount of an antitumor chemotherapeutic agent.

Preferably, the mammals treated by the methods of the present invention are humans.

In the method embodiments of the present invention, the DNA compositions can be administered enterally, such as by oral administration, or parenterally, such as by injection or intravenous infusion. Preferably, the compositions are administered orally. The compositions can be packaged in sealed containers and labeled with information useful for a clinician to effectively administer the composition.

The DNA compositions of the present invention are useful for treatment and prevention of a number of disease states. For example, a patient suffering from colorectal cancer, breast cancer, lung cancer, and the like, can benefit from immunization by the compositions of the present invention.

### BRIEF DESCRIPTION OF THE DRAWINGS

FIG. 1 is a characterization of a murine FAP DNA-construct and of chemo-resistant tumor cell lines. Panel A illustrates a plasmid cDNA encoding the entire murine FAP, inserted into the EcoRI site of the pcDNA3.1/V5-His-TOPO vector (pFAP). Panel B shows FAP protein expression was demonstrated by Western blotting after transient transfection of CT26 cells. Panel C shows CT26 colon and D2F2 breast carcinoma cells treated with various chemotherapeutic agents at the concentrations indicated. After 48 hours of incubation, nuclear apoptosis was assessed by Hoechst-33342 dye staining. Bars indicate the mean plus standard deviation of 3 assays.

FIG. 2a through FIG. 2c illustrate the effect of the FAP-based DNA composition on tumor growth. Prophylactic setting: 10 days after the last of 3 vaccinations at 1-week intervals, performed as described in Materials and Methods, BALB/c mice (n=8) were challenged by subcutaneous (s.c.) injection with a lethal dose of 3×10⁴ CT26 cells (FIG. 2a) or orthotopically (o.t.) with a lethal dose of 3×10⁵ D2F2 cells (FIG. 2b). The average tumor growth of 8 mice is depicted, mean+/−SE, P<0.01. Therapeutic setting: BALB/c mice (n=8) were first intravenously (i.v.) injected with 10⁵ CT26 cells and then treated after 3 and 10 days once pulmonary metastases were established. After 18 days, the lungs were excised and weighed (normal lung weight was about 0.2 g), examined for metastases, and scored by a visual evaluation. The percentage of lung surface covered by fused metastases was assessed as follows: 0=0%, 1=<20%, 2=20-50%, 3=>50%, P<0.01. (FIG. 2c).

FIG. 3 illustrates cytotoxicity induced by CD8⁺ T cells. (A) Depicted is the effect on life span of antibody-mediated depletion during the effector phase in treated mice (* denotes statistical significance compared to control group, p<0.01). (B) CD8⁺ T cells were purified from spleens of treated mice, stimulated with γ-irradiated tumor target cells and then incubated for 48 hours with pGFP or pGFP/pFap-transfected CT26 cells. Nuclear apoptosis was assessed by staining with Hoechst-33342 dye as follows: Nuclear apoptosis stage 0: no apoptosis; stage 1: large-scale chromatin condensation; stage 2a: chromatin fragmentation; stage 2b: apoptotic bodies. (C, D) Splenocytes from pFap and empty vector immunized mice (n=3) were stimulated for 5 days with pFap transfected A31 fibroblasts and then subjected to a ⁵¹Cr-release assay. (D) Effector and target cells were co-incubated with anti-MHC class I antibodies (Mean +SD, * denotes statistical significance compared to empty vector, p<0.05). (E) FACS analysis of single cell suspensions of CT26 tumors of treated mice (n=2) stained with anti-CD3⁺ PerCP-Cy5.5 and anti-CD8⁺ FITC antibodies. One of two experiments is depicted. (F) Representative sections of CT26 tumors of pFap and empty vector treated mice stained with anti-CD8 FITC antibodies and DAPI nuclear stain.

FIG. 4 demonstrates expression of FAP/collagen type I and intratumoral uptake of fluorescein/Albumin/5-FU. (A) Immunohistochemical analysis of FAP (upper panel) and collagen type I (lower panel) expression in s.c. CT26 tumors of treated mice. (B) Immunoblots of FAP and collagen type I in s.c. CT26 tumors of treated mice. (C, D, E) Bar graphs indicate means+SE of optical density or scintillation measurements of homogenates of s.c. CT26 tumors from treated BALB/c mice (n=4), after i.p. injection of fluorescein, i.v. injection of Evans blue albumin or i.v. injection of 14C-5-fluorouracil. P<0.05, P<0.01 and P<0.05, respectively.

FIG. 5 illustrates the anti-metastatic effects of combined bio- and chemotherapy and side effects. (A) Prophylactic setting: 10 days after the last of 3 vaccinations at 1-week intervals with control vaccine, PBS or vaccine of the invention, BALB/c mice (n=8; mean±SE) were challenged o.t. with 3×10⁵ D2F2 cells. After 5, 10 and 15 days, indicated mice were treated with doxorubicin. (B) Therapeutic setting: 5 days after i.v. injection of 105 D2F2 tumor cells BALB/c mice (n=8) were treated weekly with pFap vaccine or control vaccine. One day after each immunization mice were treated with doxorubicin i.v. as indicated (* depicts significant significance compared to control group, p<0.0001, ** depicts significance compared to control, control/Dox, pFap and pFap/Dox group, p<0.0001)

FIG. 6 (A) Intratumoral doxorubicin concentration: Treated BALB/c mice (n=4) were challenged s.c. with 5×10⁵ D2F2 cells and after 16 days the doxorubicin concentration in pooled tumor lysates was determined by LC-MS (representative of two experiments, mean +SD. P<0.001). (B) Circular wounds of 3 mm diameter were inflicted on the upper backs of treated mice (n=4) and the average time until complete wound closure was measured (mean +SD).

FIG. 7 shows the nucleotide sequence (SEQ ID NO: 1) of a nucleic acid encoding human FAP.

FIG. 8 illustrates the amino acid residue sequence of human FAP (SEQ ID NO: 2).

FIG. 9 shows the nucleotide sequence (SEQ ID NO: 3) of a nucleic acid encoding murine FAP.

FIG. 10 shows the amino acid residue sequence of murine FAP (SEQ ID NO: 4).

FIG. 11 shows the nucleotide sequence (SEQ ID NO: 5) of a nucleic acid encoding human IL-2.

FIG. 12 shows the amino acid residue sequence of human IL-2 (SEQ ID NO: 6).

FIG. 13 shows the nucleotide sequence (SEQ ID NO: 7) of a nucleic acid encoding murine IL-2.

FIG. 14 shows the amino acid residue sequence of murine IL-2 (SEQ ID NO: 8).

FIG. 15 shows the nucleotide sequence (SEQ ID NO: 9) of a nucleic acid encoding human CCL21.

FIG. 16 shows the amino acid residue sequence of human CCL21 (SEQ ID NO: 10).

FIG. 17 shows the nucleotide sequence (SEQ ID NO: 11) of a nucleic acid encoding murine CCL21b.

FIG. 18 shows the amino acid residue sequence of murine CCL21b (SEQ ID NO: 12).

FIG. 19 illustrates the amino acid sequence similarity between human CCL21 and murine CCL21b.

FIG. 20 shows the nucleotide sequence (SEQ ID NO: 13) of a nucleic acid encoding murine CCL21a.

FIG. 21 shows the amino acid residue sequence of murine CCL21a (SEQ ID NO: 14).

FIG. 22 shows the nucleotide sequence (SEQ ID NO: 15) of a nucleic acid encoding human CD40L

FIG. 23 shows the amino acid residue sequence of human CD40L (SEQ ID NO: 16).

### DETAILED DESCRIPTION OF PREFERRED EMBODIMENTS

The present invention provides a DNA composition effective for inhibiting tumor growth or tumor metastases, which targets the tumor stromal antigen known as fibroblast activating protein (FAP). The DNA composition comprises a DNA construct that encodes at least one epitope of FAP, which is expressible in immune cells, and which is incorporated in a pharmaceutically acceptable carrier. The DNA construct can encode a single epitope of FAP, a polypeptide comprising two or more epitopes of FAP, the entire FAP protein, or any portion thereof that will elicit the desired immune response against cells that express FAP, such as tumor stromal cells. The immune response elicited by the compositions of the invention leads to inhibition of tumor growth and tumor metastases.

The term “DNA construct” as used herein and in the appended claims refers to a DNA structure that encodes for a protein or polypeptide of interest, such as a FAP epitope, FAP protein, IL-2, CCL21, and the like. DNA constructs include any DNA that can be transcribed in target cells, including linear DNA and plasmid DNA, as well as DNA which has been incorporated into the genetic material of a cell or virus. Preferably, the DNA construct is a DNA that has been incorporated in a viral or bacterial delivery vector, e.g., an attenuated viral or bacterial vector that is non-pathogenic. When a subject is treated with a composition of the invention, the DNA encoding FAP is delivered to immune cells (e.g., macrophages and dendritic cells), which then express the FAP protein. Viral and bacterial carriers of the FAP DNA do not express FAP, themselves.

The DNA compositions of the present invention stimulate formation of CTLs that are active against cells that present the FAP antigen, such as tumor stromal cells (e.g., stromal fibroblast cells). Such tumor stromal cells are selectively targeted by CTLs that are produced in response to immunization by the DNA compositions of the invention. The compositions of the invention also can reduce tumor stromal expression of collagen type I, which in turn enhances uptake of chemotherapeutic agents.

As used herein, the term “immunity” refers to long term immunological protection against cells expressing an antigen. The term “immunization” refers to exposure to an antigen, which results in immunity to the cells expressing the antigen in the treated subject.

The term “FAP protein” refers to human FAP or a polypeptide that has at least 80% sequence similarity to human FAP and which includes at least one epitope of human FAP. The term “FAP DNA” refers to DNA encoding human FAP or encoding a polypeptides that has at least 80% sequence similarity to human FAP and which includes at least one epitope of human FAP.

A DNA construct useful in a DNA compositions of the present invention preferably comprises a nucleic acid that encodes a polypeptide comprising one or more epitopes of FAP, and which is operably linked to regulatory elements needed for gene expression in immune cells. Preferably, the DNA construct encodes for full length FAP protein or a polypeptide having a high degree of sequence similarity of at least 80% therewith and which includes at lease one epitope of FAP. Useful DNA constructs preferably include regulatory elements necessary for expression of nucleotides in immune cells. Such elements include, for example, a promoter, an initiation codon, a stop codon, and a polyadenylation signal. In addition, enhancers are often required for expression of a sequence that encodes an immunogenic target protein. As is known in the art, these elements are preferably operably linked to the sequence that encodes the desired protein. Regulatory elements are preferably selected that are operable in the species to which they are to be administered. Preferably, the DNA construct is in the form of a plasmid or is incorporated into a viral or bacterial vector. The DNA construct encoding the FAP protein initially can be incorporated into a bacterial vector by transfection, using methods well known in the art. Subsequently, the transformed bacteria can be cultured to provide a ready stock of bacteria, which include FAP DNA within the genetic material of the bacteria. Cultures of such transformed bacteria provide a ready source for the DNA compositions of the present invention.

Initiation codons and stop codons are preferably included as part of a nucleotide sequence that encodes FAP in a DNA composition of the present invention. The initiation and termination codons must be in frame with the coding sequence.

Promoters and polyadenylation signals included in a composition of the present invention preferably are selected to be functional within the cells of the subject to be immunized.

Examples of promoters useful in the compositions of the present invention, especially in the production of a genetic vaccine DNA composition for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV), as well as promoters from human genes, such as human actin, human myosin, human hemoglobin, human muscle creatine, and human metalothionein.

Examples of polyadenylation signals useful in the DNA compositions of the present invention, especially in the production of a DNA composition for humans, include but are not limited to SV40 polyadenylation signals and LTR polyadenylation signals.

In addition to the regulatory elements required for DNA expression, other elements can also be included in the DNA molecule. Such additional elements include enhancers. The enhancer can be, for example, human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. One having ordinary skill in the art can produce DNA constructs that are functional in a given subject species.

DNA constructs useful in the present compositions can be “naked” DNA, as defined in Restifo et al. Gene Therapy 2000; 7:89-92, the relevant disclosure of which is incorporated by reference. Preferably, the DNA construct is in the form of a plasmid or DNA that is incorporated into the genetic material of an attenuated virus or attenuated bacterium. Useful delivery vehicles or carriers include biodegradable microcapsules, immuno-stimulating complexes (ISCOMs), and liposomes for naked DNA constructs, and various physiologically acceptable buffers for genetically engineered attenuated live viruses or bacteria.

Examples of suitable attenuated live bacterial vectors that can be transformed to incorporate a FAP DNA construct include Salmonella typhimurium, Salmonella typhi, Shigella species, Bacillus species, Lactobacillus species, Bacille Calmette-Guerin (BCG), Escherichia coli, Vibrio cholerae, Campylobacter species, Listeria species, or any other suitable bacterial vector, as is known in the art. Preferably, the vector is an attenuated live Salmonella typhimurium vector, particularly when the composition is intended for oral administration. Preferred attenuated live Salmonella typhimurium include AroA⁻ strains such as SL7207, or doubly attenuated AroA⁻, dam⁻ strains, such as RE88. The doubly attenuated AroA⁻, dam⁻ Salmonella typhimurium is a particularly preferred vector.

Methods of transforming live bacterial vectors with an exogenous DNA construct are well described in the art. See, for example, Joseph Sambrook and David W. Russell, Molecular Cloning, A Laboratory Manual, 3rd Ed., Cold Spring Harbor Laboratory Press, Cold Spring Harbor, N.Y. (2001) (Sambrook and Russell). After transformation, the exogenous genetic material is incorporated into the genetic material of the bacterium, so that as the bacteria reproduce, the exogenous DNA is replicated along with the native DNA of the organism. Thus, once the bacterium has been transformed, the normal reproductive processes of the organism provides a ready supply of the exogenous DNA.

Preferred viral vectors include Bacteriophages, Herpes virus, Adenovirus, Adeno-associated virus, Sindbis virus, Polio virus, Vaccinia virus, and Avipox. Methods of transforming viral vector with an exogenous DNA construct are also well described in the art. See Sambrook and Russell, above.

Useful liposome carrier vehicles are unilamellar or multilamellar vesicles, having a membrane portion formed of lipophilic material and an interior aqueous portion. The aqueous portion is used in the present invention to contain the polynucleotide material to be delivered to the target cell. It is generally preferred that the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the relevant disclosures of which are incorporated by reference. Many other suitable liposome-forming cationic lipid compounds are described in the literature. See, e.g., L. Stamatatos et al., Biochemistry 1988; 27:3917-3925; and H. Eibl et al., Biophysical Chemistry 1979; 10:261-271. Alternatively, a microsphere such as a polylactide-coglycolide biodegradable microsphere can be utilized. A nucleic acid construct is encapsulated or otherwise complexed with the liposome or microsphere for delivery of the nucleic acid to a tissue, as is known in the art.

Other useful carrier vehicles include polymeric microspheres comprising biodegradable poly(ortho ester) materials, as described by Wang et al., Nat. Mater., 2004; 3(3):190-6. Epub 2004 Feb. 15, the relevant disclosures of which are incorporated herein by reference.

Preferably, the compositions for the present invention comprise DNA constructs that encode human FAP or a functional homolog thereof. Functional homologs of FAP preferably have at least about 80% amino acid residue sequence similarity to human FAP more preferably, at least about 90%, most preferably at least about 95% sequence similarity to human FAP.

GenBank is a genetic sequence database of the National Institutes of Health (NIH), which is an annotated collection of all publicly available DNA sequences. GenBank is part of the International Nucleotide Sequence Database Collaboration, a combined effort of the DNA DataBank of Japan (DDBJ), the European Molecular Biology Laboratory (EMBL), and GenBank at the National Center for Biotechnology Information.

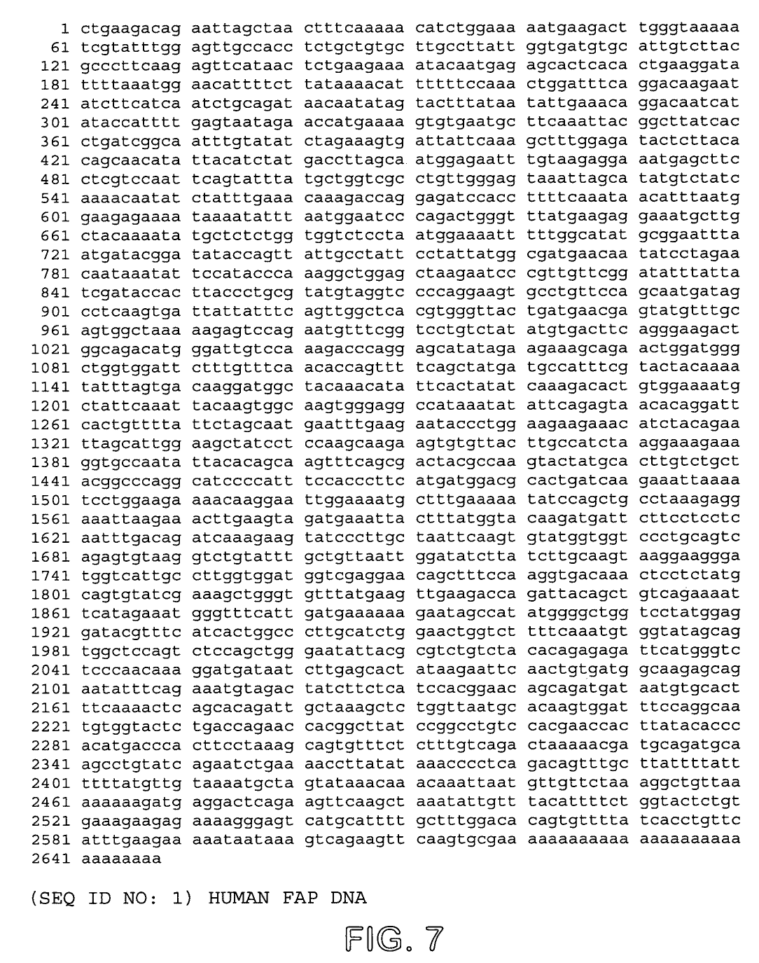

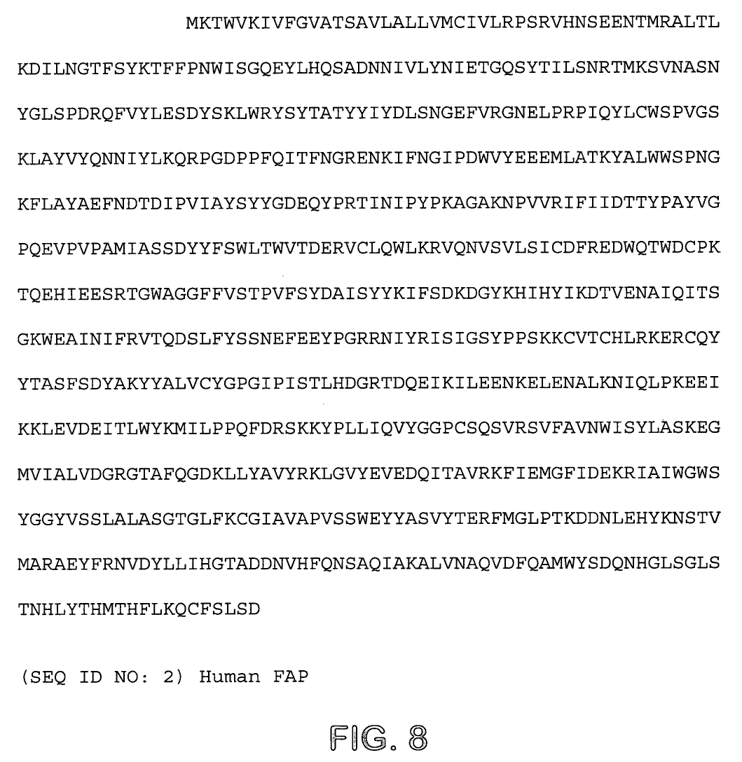

The nucleic acid sequence of a cDNA encoding human FAP, SEQ ID NO: 1 (FIG. 7) has been published in GenBank, Accession No. BC026250, the disclosure of which is incorporated herein by reference. The corresponding amino acid residue sequence of human FAP is SEQ ID NO: 2 (FIG. 8).

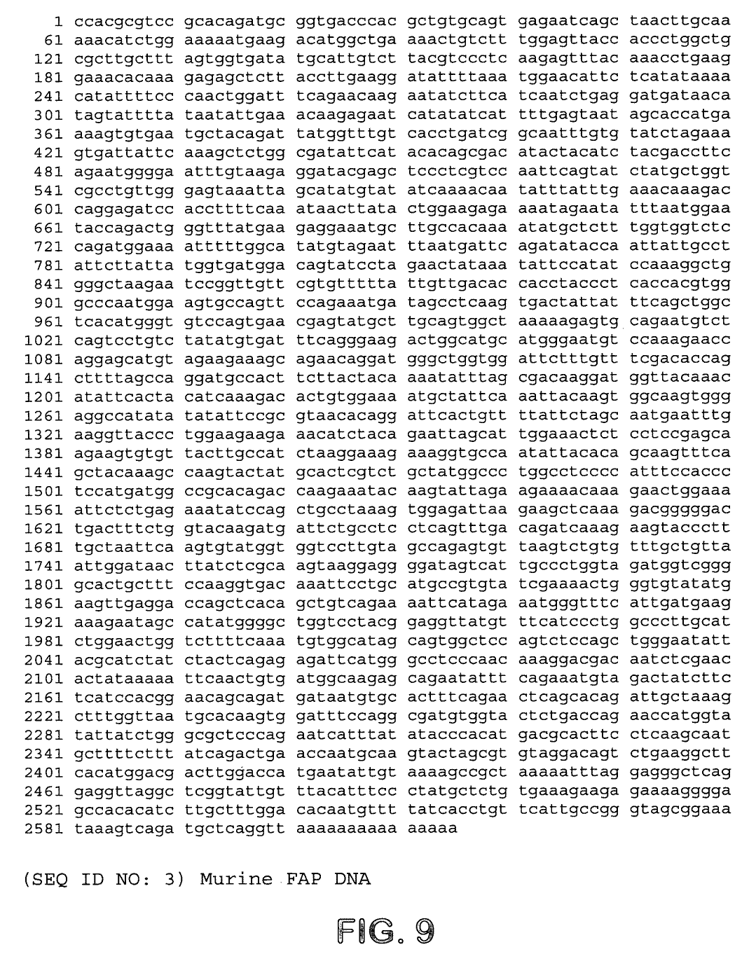

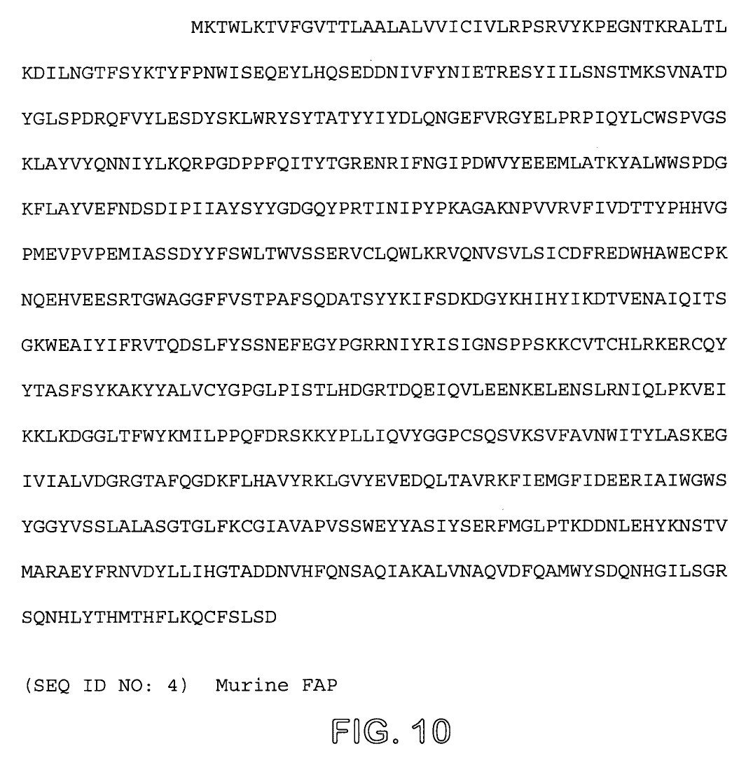

The nucleic acid sequence of a DNA encoding murine FAP, SEQ ID NO: 3 (FIG. 9) has been published in GenBank, Accession No. BC019190, the disclosure of which is incorporated herein by reference. The corresponding amino acid residue sequence of murine FAP is SEQ ID NO: 4 (FIG. 10).

Due to the inherent degeneracy of the genetic code, other DNA sequences that encode the amino acid sequence to human FAP, can be used in the practice of the invention. Such DNA sequences include those which are capable of hybridizing to human FAP as well.

DNA sequences that encode human FAP, which can be used in accordance with the invention include nucleic acids having deletions, additions or substitutions of different nucleotide residues for those in SEQ ID NO: 1, which result in a sequence that encodes the same FAP gene product. DNA molecules encoding for functionally equivalent homologs of human FAP can also be used in the DNA compositions of the present invention.

The gene product encoded by nucleic acid may also contain deletions, additions or substitutions of amino acid residues within the FAP amino acid residue sequence, which results in a silent change, thus producing a functionally equivalent FAP. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. As used herein, a functionally equivalent FAP refers to protein that includes one or more epitopes, which when recognized by T cells, allow these same T cells to recognize FAP epitopes displayed on FAP-expressing cells. In preferred embodiments, a functionally equivalent FAP has an amino acid residue sequence that has at least about 80% sequence similarity to the amino acid residue sequence of human FAP (SEQ ID NO: 2), e.g., at least about 90% sequence similarity or at least about 95% sequence similarity to human FAP.

The DNA constructs encoding FAP can be engineered in order to alter the FAP coding sequence (relative to the native FAP cDNA, SEQ ID NO: 1) for a variety of ends including, but not limited to, alterations that modify processing and expression of the FAP gene product. For example, mutations may be introduced in the DNA using techniques that are well known in the art, e.g., site-directed mutagenesis, to insert new restriction sites, to alter glycosylation patterns, phosphorylation, etc.

In one preferred embodiment, the DNA composition of the invention comprises a DNA construct encoding FAP and a DNA construct operably encoding at least one immune effector protein, both of which are expressible in immune cells. As used herein and in the appended claims the phrase “immune effector protein” means a protein that is involved in regulation of an immune system pathway. Preferably, the immune effector protein is a cytokine.

Cytokines are proteins and polypeptides produced by cells that can affect the behavior of other cells, such as cell proliferation, cell differentiation, regulation of immune responses, hematopoiesis, and inflammatory responses. Cytokines have been classified into a number of families, including chemokines, hematopoietins, immunoglobulins, tumor necrosis factors, and a variety of unassigned molecules. See generally Oxford Dictionary of Biochemistry and Molecular Biology, Revised Edition, Oxford University Press, 2000; and C. A. Janeway, P. Travers, M. Walport and M. Schlomchik, Immunobiology, Fifth Edition, Garland Publishing, 2001 (hereinafter “Janeway and Travers”). A concise classification of cytokines is presented in Janeway and Travers, Appendix III, pages 677-679, the relevant disclosures of which are incorporated herein by reference.

Hematopoietins include, for example erythropoietin, interleukin-2 (IL-2, a 133 amino acid protein produced by T cells and involved in T cell proliferation), IL-3, IL-4, IL-5, IL-6, IL-7, IL-9, IL-11, IL-13, IL-15 (a 114 amino acid IL-2-like protein, which stimulates the growth of intestinal epithelium, T cells, and NK cells), granulocyte colony-stimulating factor (G-CSF), granulocyte-macrophage colony-stimulating factor (GM-CSF), oncostatin M (OSM), and leukemia inhibitory factor (LIF).

Interferons include, for example, IFN-α, IFN-β, and IFN-γ (a 143 amino acid homodimeric protein produced by T cells and NK cells, which is involved in macrophage activation, increased expression of MHC molecules and antigen processing components, IG class switching, and suppression of T_{H}2).

Immunoglobulins include, for example, B7.1 (CD80), and B7.2 (CD86), both of which co-stimulate T cell responses.

The tumor necrosis factor (TNF) family includes, for example, TNF-α, TNF-β (lymphotoxin), lymphotoxin-β(LT-β), CD40 ligands, Fas ligand, CD27 ligand, CD30 ligand, 4-1BB ligand, Trail, and OPG ligand.

The biological roles of CD40 ligand (CD40L), particularly its interaction with CD40 expressed on antigen presenting cells during costimulation of T cell activation, are well known in the art. CD40 is a 48 kDa glycoprotein expressed on the surface of all mature B cells, most mature B-cell malignancies, and some early B-cell acute lymphocytic leukemias, but it is not expressed on plasma cells, Clark, Tissue Antigens 1990, 35:33-36. CD40L, a type II membrane protein of about 35 kDa, is expressed on the surface of T cells upon antigen recognition. Members of the TNF family are biologically most active when expressed as homotrimers. CD40L is no exception in this regard and can be expressed as a homotrimer (CD40LT) by modification of a 33 amino acid leucine zipper motif fused to the N-terminus of the entire extracellular domain of this ligand. CD40LT DNA has been reported by Gurunathan et al. J. Immunol. 1998, 161:4563, to enhance cellular immune responses such as induction of IFN-γ and cytolytic T cell activity when mice were treated with DNA encoding the highly immunogenic model antigen β-galactosidase.

CD40LT is an important factor in the activation of T cells necessary to induce an effective protective immunity against tumor self-antigens. Once antigen loaded MHC class I complexes are taken up by dendritic cells (DCs) and presented to naive T cells, the first antigen signal is delivered via T cell receptors (TCR), followed by upregulation of CD40LT. On the T cell surface, CD40LT then induces costimulatory activity on DCs via CD40-CD40LT interactions. Thus primed, these APCs can express costimulatory molecules B7.1 (CD80) and B7.2 (CD86), which sends a second costimulatory signal to T cells via interaction with CD28, an event that leads to full activation of T cells to concurrently produce pro-inflammatory cytokines INF-γ and IL12, and to perform effector functions.

Various cytokines that are not assigned to a particular family include, for example, tumor growth factor-β (TGF-β), IL-1α, IL-1β, IL-1 RA, IL-10, IL-12 (natural killer cell stimulatory factor; a heterodimer having a 197 amino acid chain and a 306 amino acid chain, which is involved in NK cell activation and induction of T cell differentiation to T_{H}1-like cells), macrophage inhibitory factor (MIF), IL-16, IL-17 (a cytokine production-inducing factor, which induces cytokine production in epithelia, endothelia, and fibroblasts), and IL-18.

Chemokines are a family of cytokines that are relatively small chemoattractant proteins and polypeptides, which stimulate the migration and activation of various cells, such as leucocyte migration (e.g., phagocytes and lymphocytes). Chemokines play a role in inflammation and other immune responses. Chemokines have been classified into a number of families, including the C chemokines, CC chemokines, CXC chemokines, and CX₃C chemokines. The names refer to the number and spacing of cysteine (C) residues in the molecules; C chemokines having one cysteine, CC chemokines having two contiguous cysteines, CXC having two cysteines separated by a single amino acid residue, and CX₃C chemokines having two cysteines separated by three amino acid residues. Chemokines interact with a number of chemokine receptors present on cell surfaces. See Janeway and Travers, Appendix IV, page 680, which is incorporated herein by reference.

In addition, chemokines can have immunomodulating activity and have been implicated in immune responses to cancer. For example, murine 6Ckine/SLC, the mouse analog of the human secondary lymphoid tissue chemokine (SLC), now commonly referred to as CCL21, has been reported to induce an antitumor response in a C26 colon carcinoma tumor cell line. See Vicari et al. J. Immunol. 2000; 165(4):1992-2000. Human CCL21 and its murine counterpart, 6Ckine/SLC, are classified as CC chemokines, which interact with the CCR7 chemokine receptor. Murine 6Ckine/SLC (muCCL21) is also reported by Vicari et al. to be a ligand for the CXCR3 chemokine receptor. Human CCL21, murine muCCL21, and a variety of other chemokines are implicated in the regulation of various immune system cells such as dendritic cells, T cells, and natural killer (NK) cells.

Mig and IP-10 are CXC chemokines that interact with the CXCR3 receptor, which is associated with activated T cells. Lymphotactin is a C chemokine, which interacts with the XCR1 receptor associated with T cells and NK cells. Fractalkine is a CX₃C chemokine, which interact with the CX₃CR1 receptor that is associated with T cells, monocytes and neutrophils.

Particularly preferred immune effector proteins to be encoded by the DNA compositions of the present invention include cytokines IL-2 (a hematopoietin), CCL21 (a chemokine), as well as CD40 ligands such as CD40 ligand trimer (CD40LT), a TNF family cytokine.

DNA and protein sequences for human IL-2 have been published in GenBank, Accession No. BC070338, the disclosures of which are incorporated herein by reference. The DNA and protein sequences of murine IL-2 have been in GenBank, Accession No. NM 008366, the disclosures of which are incorporated herein by reference.

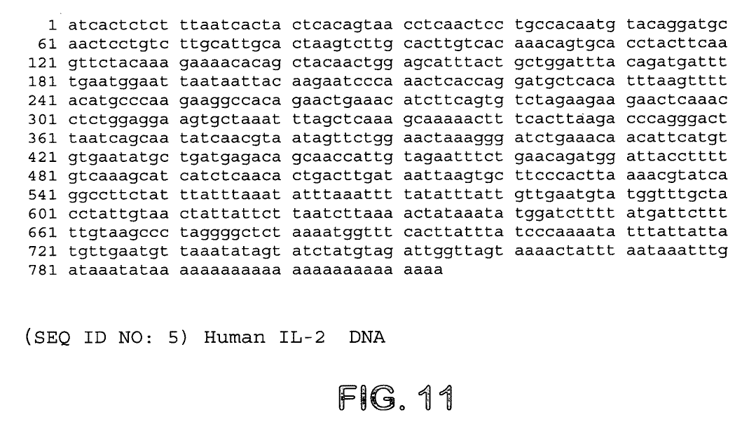

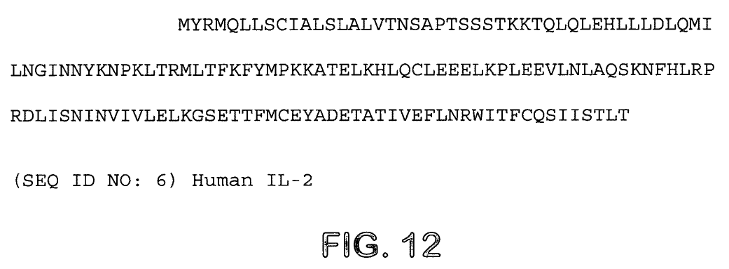

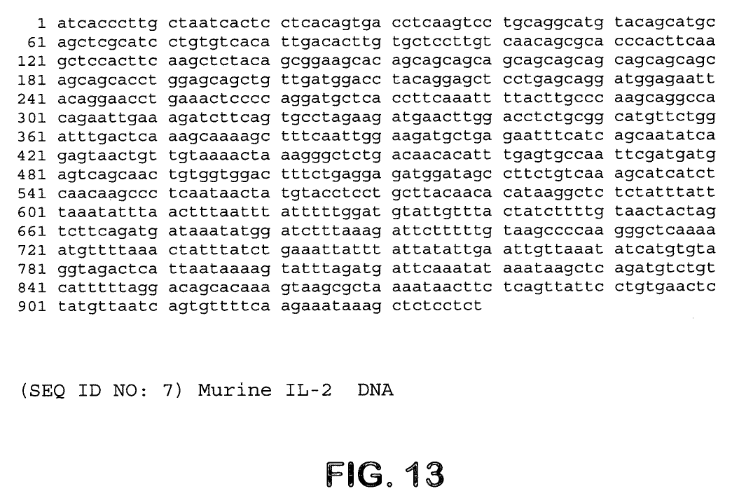

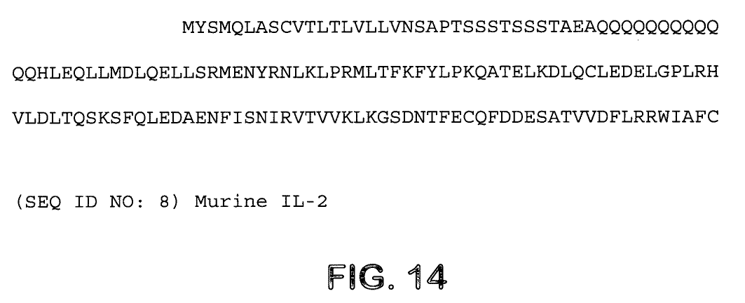

The nucleic acid sequence encoding human IL-2 is presented in FIG. 11 (SEQ ID NO: 5), and its corresponding amino acid residue sequence (SEQ ID NO: 6) is provided in FIG. 12. The nucleic acid sequence encoding murine IL-2 is presented in FIG. 13 (SEQ ID NO: 7), and its corresponding amino sequence (SEQ ID NO: 8) is provided in FIG. 14.

DNA and protein sequences for human CCL21 have been published in GenBank, Accession No. AB002409, the disclosures of which are incorporated herein by reference. The DNA and protein sequences of the murine CCL21a variant have been published in GenBank, Accession No. NM011335, the disclosures of which are incorporated herein by reference. The DNA and protein sequences of the murine CCL21b variant have been published in GenBank, Accession No. NM011124, the disclosures of which are incorporated herein by reference.

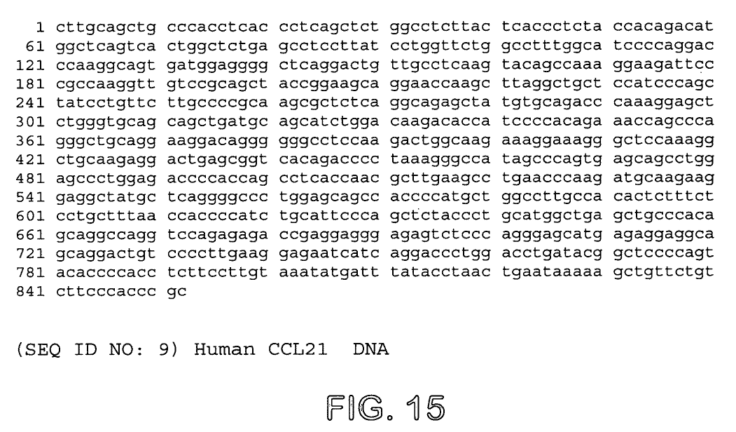

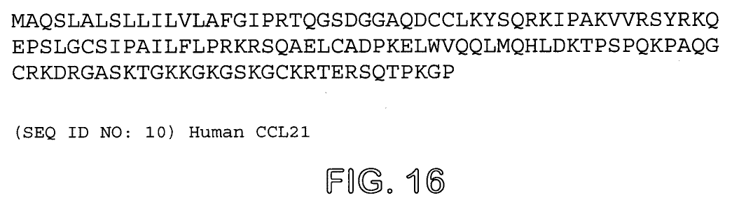

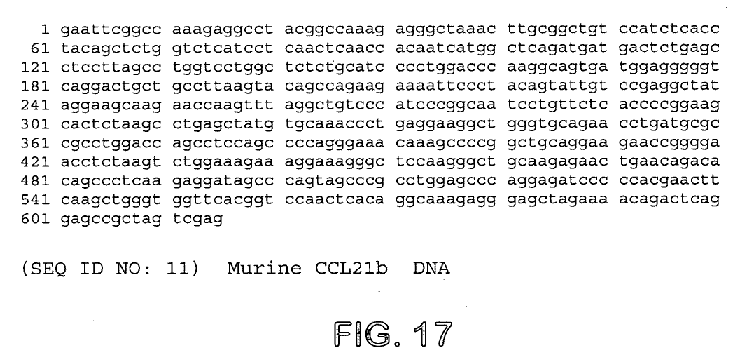

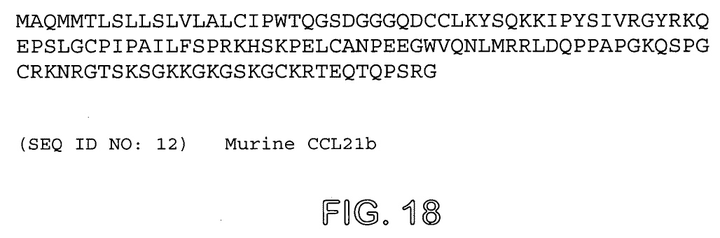

The nucleic acid sequence encoding human CCL21 is presented in FIG. 15 (SEQ ID NO: 9), and its corresponding amino acid residue sequence (SEQ ID NO: 10) is provided in FIG. 16. The nucleic acid sequence encoding murine CCL21 (CCL21b variant) is presented in FIG. 17 (SEQ ID NO: 11), and its corresponding amino sequence (SEQ ID NO: 12) is provided in FIG. 18.

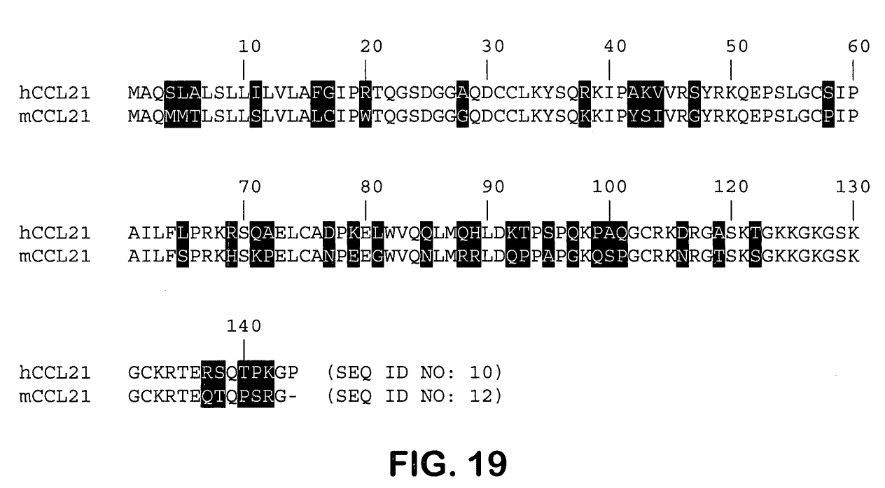

The protein sequence similarity between human CCL21 and its murine counterpart (murine CCL21b) is illustrated in FIG. 19. There is about 73% amino acid residue sequence identity between human CCL21 (SEQ ID NO: 10) and murine CCL21b (SEQ ID NO: 12).

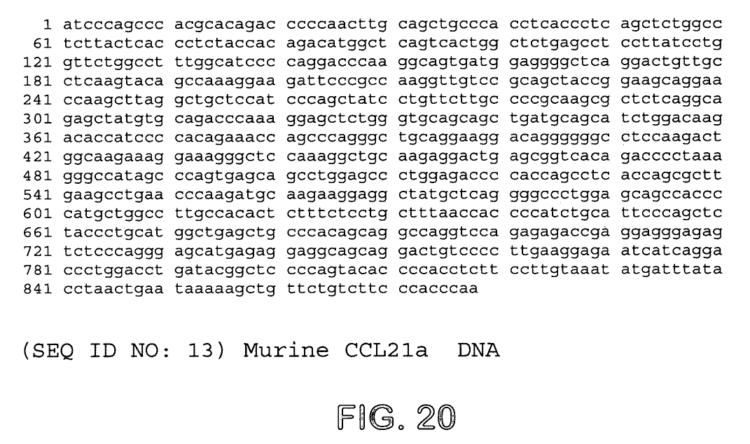

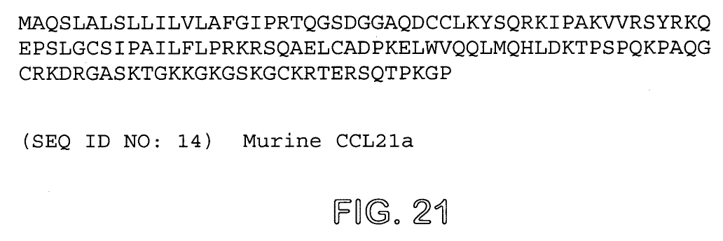

The nucleic acid sequence encoding the CCL21a variant of murine CCL21 is presented in FIG. 20 (SEQ ID NO: 13), and its corresponding amino sequence (SEQ ID NO: 14) is provided in FIG. 21.

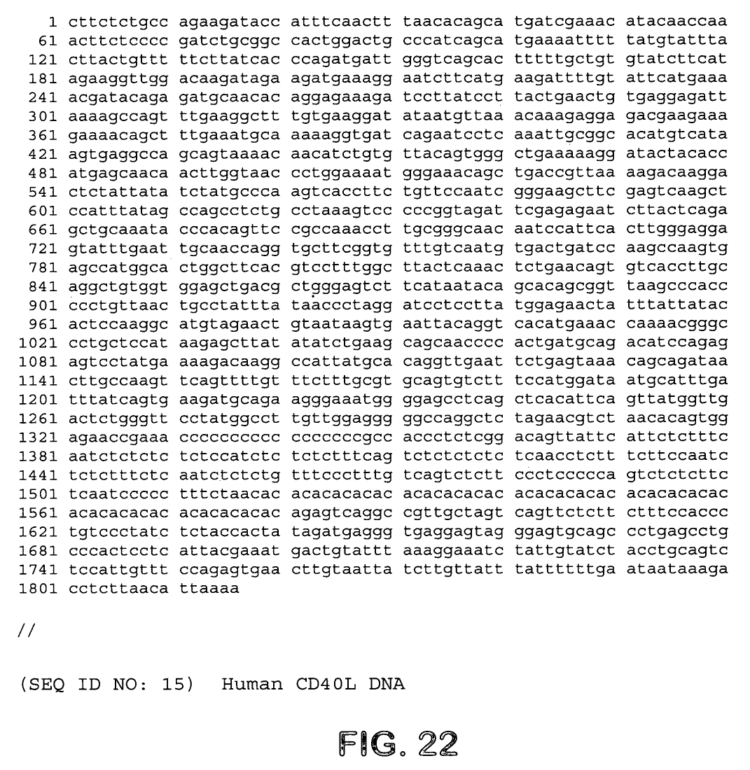

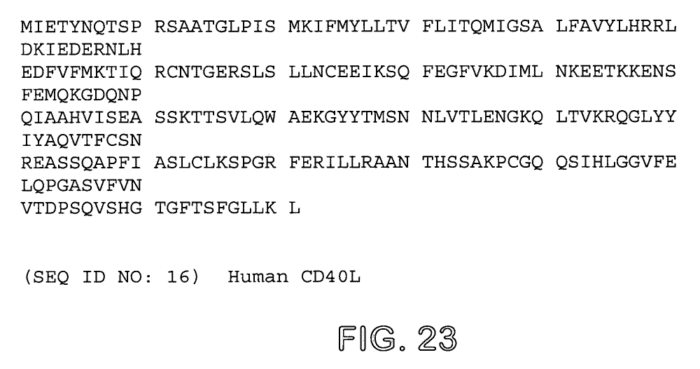

Human CD40 ligand (CD40L) is a 261 amino acid protein, which exists as a trimer (CD40LT) in its most active form. The DNA sequence encoding human CD40L (also known as CD154) has been published in GenBank, Accession No. NM 000074, the disclosure of which is incorporated herein by reference (FIG. 22, SEQ ID NO: 15). The corresponding protein sequence of CD40L is shown in FIG. 23 (SEQ ID NO: 16).

The method aspects of the present invention involve administering to a mammal a DNA composition comprising a DNA construct encoding FAP, which is expressible in immune cells of the mammal. Preferably, the mammal is a human. The composition can be administered orally, intramuscularly, intranasally, intraperitoneally, subcutaneously, intradermally, or topically, depending upon the particular dosage form in which the composition is prepared. Preferably the composition is prepared in an orally administrable dosage form, such as a solution, suspension, emulsion, capsule, tablet, and the like.

A DNA composition of the invention can be utilized to provide long term inhibition of tumor growth and/or tumor metastases in a patient treated with the composition. In a preferred embodiment, the DNA composition is administered in conjunction with an antitumor chemotherapeutic agent. The DNA composition can be administered together with the chemotherapeutic agent in a combined dosage form, or the composition and chemotherapeutic agent can be administered in separate dosage forms and at separate dosage intervals tailored to the pharmacology of the chemotherapeutic agent being administered.

Chemotherapeutic agents useful in combination with the DNA compositions of the present invention include antitumor agents such as doxorubicin, paclitaxol, a cyclophosphamide, etoposide, 5-fluorouracil, methotrexate, and the like.

DNA compositions of the present invention preferably are formulated with pharmaceutically acceptable carriers or excipients such as water, saline, dextrose, glycerol, and the like, and combinations thereof to aid in formulation and administration of the composition. The compositions can also contain auxiliary substances such as wetting agents, emulsifying agents, buffers, and other auxiliary substances that are well known in the pharmaceutical arts.

The compositions of the present invention are preferably administered orally to a mammal, such as a human, as a solution or suspension in a pharmaceutically acceptable carrier, at a DNA concentration in the range of about 1 to about 10 micrograms per milliliter, based on the weight of the DNA that encodes FAP. A particularly preferred dosage form for a DNA composition of the invention is a suspension of attenuated FAP-transfected bacteria in a suitable buffer solution, which can be formulated for oral administration. The appropriate dosage of the composition will depend upon the subject to be treated, on the activity of the composition, and in part upon the judgment of the medical practitioner administering or requesting administration of the composition.

The dosage to be administered to the mammal and the schedule of administration if more than one administration is to be used, will vary from mammal to mammal and upon the dosage form. Effective dosage amounts and administration schedules can be determined empirically through clinical dose-response studies, as is well known in the art. The dosage and dosage schedule is selected to provide a sufficient amount of FAP expression in immune cells to elicit an immune response in the mammal against cells that present the FAP antigen. Preferably, the dosage of the composition administered to the subject mammal elicits expression of a sufficient amount of FAP antigen in immune cells of the mammal to sustain an immune response against FAP-presenting cells that will continue over a period of at least a month, e.g., at least 6 months or at least about one year.

The compositions of the present invention can be packaged in suitably sterilized containers such as ampules, bottles, or vials, either in multi-dose or in unit dosage forms. The containers are preferably hermetically sealed after being filled with a DNA composition of the invention. Preferably, the compositions are packaged in a container having a label affixed thereto, which label identifies the composition, and bears a notice in a form prescribed by a government agency such as the United States Food and Drug Administration reflecting approval of the composition under appropriate laws, dosage information, and the like. The label preferably contains information about the composition that is useful to an health care professional administering the composition to a patient. The package also preferably contains printed informational materials relating to the administration of the composition, instructions, indications, and any necessary required warnings.

The following examples are provided to further illustrate the features and embodiments of the present invention, and are not meant to be limiting.

### Materials, Methods and Examples.

Animals, bacterial strains, and cell lines. Female BALB/c mice, 6-8 weeks of age, were obtained from the Scripps Research Institute (TSRI) Rodent Breeding Facility. All animal experiments were performed according to the National Institutes of Health Guide for the Care and Use of Laboratory Animals and approved by the TSRI Animal Care Committee. The double attenuated Salmonella typhimurium strain RE88 (AroA⁻ dam⁻) was provided by Remedyne Corporation (Santa Barbara, Calif.). Murine D2F2 breast carcinoma cells were obtained from Dr. Wei-Zen Wei, Karmanos Cancer Center, Detroit, Mich., and CT26 colon carcinoma cells were obtained from ATCC (Manassas, Va.) and then cultivated over several months to obtain chemoresistant subclones.

Murine CT26 and D2F2 carcinoma cells were cultured in the presence of either 1% DMSO, etoposide, 5-fluorouracil, doxorubicin, vinblastine or paclitaxel (Sigma, St. Louis, Mo.) at the concentrations indicated. After 48 hours, apoptotic nuclei were counted after incubation with the DNA-specific Hoechst-33342 dye (2 μM; Molecular Probes, Eugene, Oreg.).

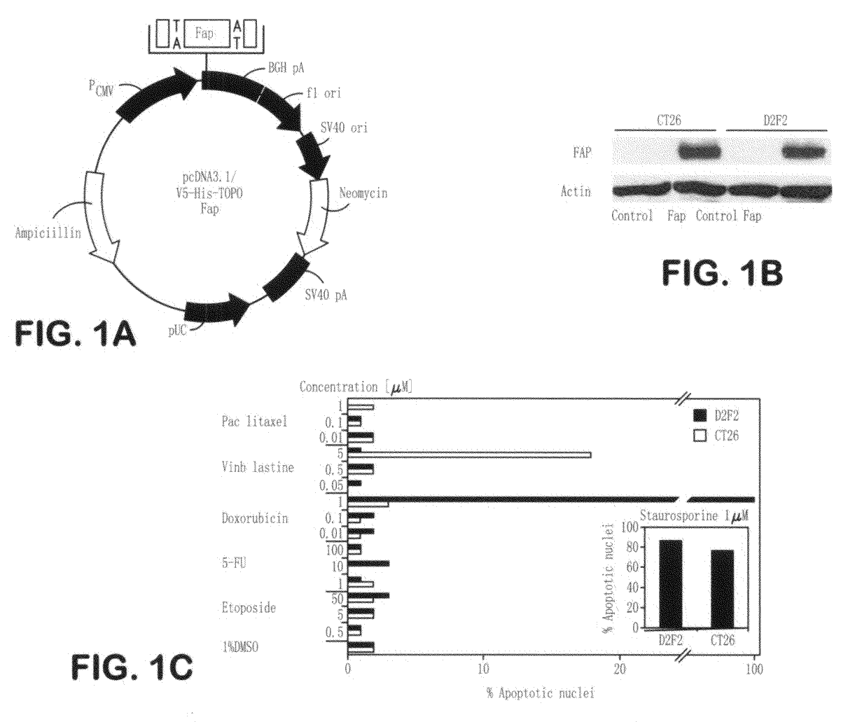

Two clones of CT26 colon cancer and D2F2 breast cancer cells, respectively, have acquired chemoresistance (see FIG. 1, Panel C). Upon addition of five different apoptosis-inducing chemotherapeutic agents to the CT26 colon carcinoma clone (FIG. 1, Panel C), vinblastine alone was able to induce a slight apoptotic effect, however only at the very high concentration of 5 μM. The original parent CT26 cell line (ATCC #CLR-2326) had an IC₅₀ for 5-fluorouracil (5-FU) of about 1.85 μM, whereas 5-FU is not able to induce nuclear apoptosis even at a concentration as high as 100 μM in the resistant CT26 clone. In the resistant D2F2 breast carcinoma cells, only doxorubicin at the highest concentration of 1 μM was able to induce nuclear apoptosis as determined by Hoechst-33342 dye staining. These results demonstrate that both of these tumor cell lines are multi-drug resistant.

Statistical analysis. The statistical significance of differential findings between experimental groups and controls was determined by Student's t-test. Significance of metastasis scores were determined by the Mann Whitney U-test. Significance of survival data was determined by the log-rank test. Findings were regarded significant, if P values were <0.05.

### Example 1

### Preparation of DNA Composition 1 Encoding Murine FAP

A cDNA (SEQ ID NO: 3, FIG. 9; provided by Dr. J. D. Cheng) encoding murine FAP (SEQ ID NO: 4, FIG. 10), was subcloned into the EcoRI restriction site of the pcDNA3.1/V5-His-TOPO vector (Invitrogen, San Diego, Calif.), to afford the eukaryotic expression vector pcDNA3.1-FAP (pFAP) (see FIG. 1, Panel A). The correct expression of the 95 kDa FAP protein from the vector was demonstrated by Western-blotting of cell lysates from transiently transfected CT26 colon carcinoma cells, which do not express FAP by themselves (FIG. 1, Panel B). This was also the case for transiently transfected D2F2 breast carcinoma cells. For transient transfections, CT26 and D2F2 cells were electroporated as described previously (See Loeffer et al., FASEB, J. 2001, 15: 758-767, incorporated herein by reference). Protein expression of FAP was demonstrated by Western blotting with a polyclonal rabbit anti-murine FAP antibody (provided by Dr. J. D. Cheng).

DNA composition 1 was prepared as follows: the pcDNA3.1-FAP vector (about 2 μg of pDNA) was electroporated into freshly prepared doubly attenuated Salmonella typhimurium (strain RE88), utilizing a Bio-Rad Pulser at 2 kV, 960 μF, and 200 Ohm according to the manufacturer's recommended procedures. Attenuated Salmonella typhimurium containing the vector were selected on ampicillin-containing plates. Colonies were picked the next day and cultured overnight in Luria-Bertani (LB) broth (10 grams tryptone, 5 grams yeast extract, and 10 grams sodium chloride in 1 L of deionized water; EM Science, Gibbstown, N.J.) with ampicillin added. The bacteria were isolated and washed in phosphate buffered saline (PBS). The washed bacteria were then suspended in PBS medium at a concentration of about 10⁹ recombinant Salmonella per milliliter of PBS, to form a solution of DNA Composition 1 containing a DNA construct that operably encodes for murine FAP, incorporated in an attenuated Salmonella typhimurium (AroA⁻ dam⁻) vector. The composition was stored in sealed ampules until used. A “control vaccine” consisting of Salmonella transformed with the pcDNA3.1 vector alone (no FAP DNA) was also prepared according to the same procedure. The plasmid DNA was stored at about −80° C. before transforming the Salmonella.

After constructing the eukaryotic expression vector pcDNA3.1/V5-His-TOPO-Fap (pFap) as described above (FIG. 1, Panel A), the correct expression of the 88 kDa FAP protein was demonstrated by Western-blotting of cell lysates from both, transiently transfected CT26 colon carcinoma and D2F2 breast carcinoma cells, which themselves do not express FAP (FIG. 1, Panel B).

### Example 2

### Preparation of DNA Composition 2 Encoding Murine FAP, Murine IL-2, and Murine CCL21

cDNAs encoding murine IL-2 (DNA SEQ ID NO: 5), from ATCC, Accession #39892, Manassas, Va., and murine CCL21a (DNA SEQ ID NO: 7) from Invitrogen, San Diego, Calif., were subcloned into the murine pFAP vector of Example 1, by the same general procedure described in Example 1. RE88 Salmonella typhimurium were transformed with the resulting vector (pFAP/IL-2/CCL21) by the procedure described in Example 1 to provide a solution of DNA Composition 2 containing a DNA construct that operably encodes murine FAP, murine IL-2, and murine CCL21a, incorporated in an attenuated Salmonella typhimurium vector.

### Example 3

### Evaluation of DNA Compositions of the Invention in Murine Models of Colon and Breast Cancer

Oral immunization, tumor cell challenge and treatment with doxorubicin. For experiments in a prophylactic setting, BALB/c mice (n=8) were treated three times at about 1-week intervals by oral gavage with about 100 μl PBS containing about 10⁹ S. typhimurium (AroA⁻ dam⁻) transformed with plasmid vectors encoding murine FAP (DNA Composition 1 of Example 1), murine FAP, IL-2 and CCL21 (DNA Composition 2 of Example 2), or control vaccine of Example 1, by methods described in Niethammer et al., Nat. Med., 2002, 8: 1369-1375. Animals were challenged about 10 days later by subcutaneous (s.c.) injection of about 3×10⁴ CT26 colon carcinoma cells in the left front flank or by orthotopic (o.t.) injection of about 3×10⁵ D2F2 breast cancer cells in the left, second lowest mammary fat pad.

Tumor volume (in mm³) was calculated by measuring the tumor in 2 dimensions (i.e., length and width, in millimeters) and calculating the volume as one half the length times the square of the width. In the therapeutic setting, the initial tumor cell inoculation was by intravenous (i.v.) injection of about 1×10⁵ CT26 colon carcinoma cells followed about 3 and 10 days later by oral vaccinations with control vaccine or active DNA compositions. After about 18 days, the lungs were weighed (normal lung weight was about 0.2 g) and pulmonary tumor metastases were examined and scored by visual evaluation assessing the percentage of lung surface covered by fused metastases as follows: score of 0 for 0% coverage, score of 1 for less than about 20% coverage, score of 2 for about 20-50% coverage, score of 3 for greater than about 50% of lung surface covered. Treatment with doxorubicin was performed in groups of mice with about 10 mg/kg doxorubicin (Sigma) administered i.v., 5, 10 and 15 days after tumor challenge.

For the depletion of CD4⁺ and CD8⁺ T cells or NK cell subpopulations, antibodies (500 μg) directed against either CD4 (clone GK 1.5) or CD8 (clone 2.43), both from National Cell Culture Center (Minneapolis, Minn.), or anti-asialo GM1 antibody (Wako BioProducts, Richmont, Va.) were injected i.p. every 7 days starting one day before tumor cell challenge.

Cytotoxicity of CD8⁺ T cells. About 10 days after the last of three treatments at 1 week intervals, splenocytes from the various treated groups of BALB/c mice (n=4) were collected. Using CD8a-micro beads (Miltenyi Biotech, Bergisch Gladbach, Germany), CD8⁺ cells were purified according to the manufacturer's protocol. These cells were then stimulated in a 5-day co-culture with γ-irradiated (1000 Gy, 45 minutes) CT26 cells were transiently transfected with either pcDNA3.1/Zeo (empty vector of Example 1) or pcDNA3.1/Zeo-FAP (pFAP vector of Example 1). Thereafter, the CD8⁺ T cells were co-cultivated with CT26 carcinoma cells (E:T=100:1) which had been transiently transfected with either green fluorescent protein (GFP), (PEGFP; Clontech, Palo Alto, Calif.) as a control or GFP plus pFAP plasmid encoding murine FAP. After about 48 hours, nuclear apoptosis was assessed as described above with the DNA-specific Hoechst 33342 dye (2 μM).

For the ⁵¹Cr release assay Balb/c mice (n=3) were treated four times at weekly intervals. Splenocytes were harvested 13 days after the last treatment and incubated for 5 days with γ-irradiated (about 1000 Gy, 45 minutes) A31 fibroblasts (ATCC, Manassas, Va.), which were retrovirally infected with pFap. Stimulated splenocytes were then incubated for about 4 hours with labeled A31-pFap and the percentage of lysis was calculated. Cells were also co-incubated with anti-MHC class I antibodies (BD Biosciences, Rockville, Md.) at a concentration of about 10 μg/ml.

Immunohistochemistry. Cryosections (about 8 μm thickness) were fixed in acetone and strained. After incubation with the primary antibody (either a polyclonal rabbit anti-murine FAP antibody (provided by Dr. J. D. Cheng), or a polyclonal rabbit anti-murine collagen type I antibody (Chemicon, Temecula, Calif.), the sections were immunostained according to the manufacturer's protocol (DAKO LSAB+ Kit, Peroxidase, DAKO, Carpinteria, Calif.).

For focal microscopy, fixed cryosections were stained with anti-murine CD8 antibody, biotinated anti-rat Ig secondary antibody, FITC labeled Streptavidin (BD Bioscience, Rockville, Md.) and co-stained with DAPI (Sigma, St. Louis, Mo.). A laser scanning confocal microscope (LSCM) was used to obtain images, which were processed using Zeiss Image Examiner software (Carl Zeiss). For FACS analysis of T-cell infiltration Balb/c mice (n=6) were treated three times at weekly intervals with either pFap or empty vector. One week after the last treatment animals were challenged s.c. with about 3×10⁵ CT26 tumor cells. Tumors were harvested 3 weeks later and single cell suspensions prepared by incubating sliced tumor tissue for 45 minutes in medium supplemented with collagenase type I (125 U/ml; GIBCO, Gaithersburg, Md.). After filtering, cells of two mice were pooled, stained with anti-CD3⁺ PerCp-Cy5.5 and anti-CD8⁺ FITC (BD Biosciences, Rockville, Md.) and analyzed by FACS.

Intratumor uptake of fluorescein, Evans blue albumin, and ¹⁴C-5-fluorouracil. After the last of three treatments at 1-week intervals with either control vaccine (Example 1), DNA Composition 1 (Example 1) or DNA Composition 2 (Example 2), mice were challenged 10 days later by subcutaneous injection of about 3×10⁴ CT26 cells in the left front flank. About 19 days thereafter, mice were given intrapertioneal (i.p.) injections of either 1% fluorescein sodium (Sigma) at about 12 μl/g of body weight, i.v. injection of about 100 μl Evans blue albumin (Sigma), or i.v. injection of about 2.5 μCi of ¹⁴C-5-fluorouracil (Sigma). After about 5 minutes, 30 minutes or 1 hour, respectively, mice were sacrificed to determine absorption or scintillation of the supernatants of tumor homogenates at 490 nm, 612 nm, or in a γ-counter, respectively.

For the determination of intratumoral doxorubicin uptake Balb/c mice (n=4) were challenged s.c. with about 5×10⁵ D2F2 cells in the right flank. Doxorubicin was injected i.v. 16 days later (10 mg/kg) and tumors were harvested 45 minutes thereafter. Samples were measured against the internal standard daunorubicin using an Eclipse XCB-C8 column on a 1100 series LC-MS (Agilent, Foster City, Calif.).

Evaluation of potential side effects. In order to determine any detrimental effects on wound healing, mice were surgically wounded. A circular wound of about 3 mm in diameter was made with a dermal punch (Miltex Inc., Bethpage, N.Y.) on the upper back of BALB/c mice (n=4) about 10 days after the last of 3 treatments at 1-week intervals. The time required for wound closure was measured. Despite the fact that FAP is overexpressed during wound healing, the compositions of the present invention do not interfere with the wound healing process. After inflicting a circular wound of about 3 mm diameter on the backs of treated BALB/c mice (n=4), no significant differences in wound healing was observed between treated and non-treated mice. For histological analysis wounds were inflicted on treated mice 7, 14, 21 days before examination. Skin biopsies, as well as 26 organs and tissues were examined by a mouse pathologist.

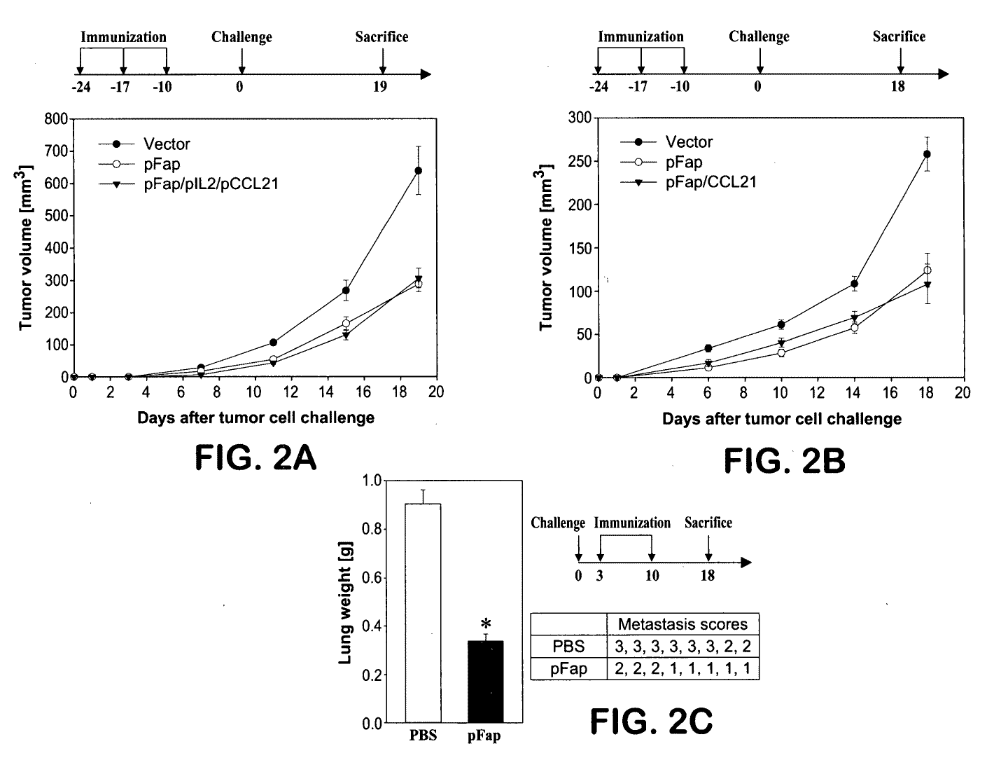

Prophylactic treatment with the FAP-based DNA-composition inhibits primary tumor growth. Ten days after the last of three treatments at one week intervals with either control vaccine (Example 1), DNA Composition 1 (Example 1) or DNA Composition 2 (Example 2), different groups of BALB/c mice (n=8) were challenged either subcutaneously with CT26 colon carcinoma cells or orthotopically with D2F2 breast carcinoma cells as described above. The DNA compositions of the invention suppressed primary tumor growth of multi-drug resistant CT26 cells (FIG. 2a), as well as the resistant D2F2 cells (FIG. 2b). DNA Composition 2, which encodes for a combination of the FAP, CCL21, and IL-2, were about equally effective in inhibiting primary tumor growth (FIG. 2a).

A DNA composition of the invention reduces growth of established metastasis in a therapeutic setting. BALB/c mice treated with DNA Composition 1 of Example 1 three and ten days after an initial intravenous inoculation with about 1×10⁵ CT26 colon carcinoma cells resulted in a significant reduction in experimental pulmonary metastases compared to mice treated with control vaccine of Example 1. In contrast, mice in the control group exhibited extensive metastases and began to die 18 days after tumor cell inoculation (FIG. 2c). Similarly, a combination of pFAP with pCCL21 alone was also about equally effective (FIG. 2b).

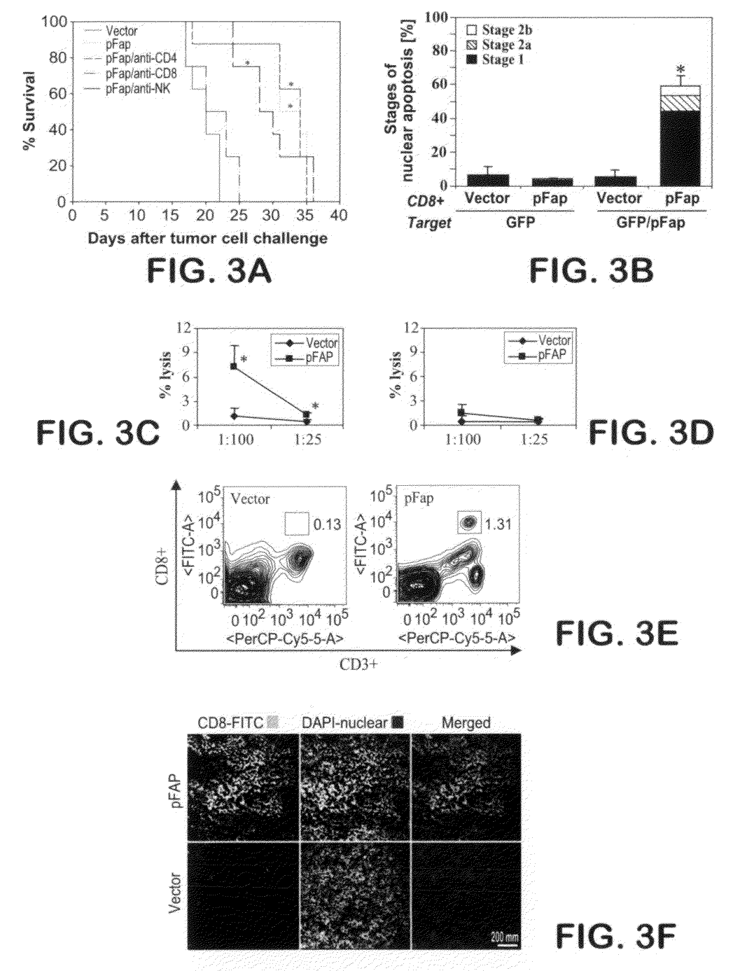

CD8⁺ T cells provide an effective anti-tumor immune response. Mice were treated i.v. three times at 1-week intervals with DNA Composition 1 (Example 1) and challenged with about 1×10⁵ CT26 cells. The mice were then depleted of their respective effector cells during the effector phase with antibodies against CD4⁺ or CD8⁺ T cells, as well as NK cells (FIG. 3, Panel A). Depletion of CD4⁺ cells and NK cells did not decrease the effectiveness of the pFAP treatment, indicating an important role for CD8⁺ T cells in the immune response.

To assess the extent to which treatment with the FAP DNA compositions of the invention is able to break peripheral T cell tolerance against the FAP self-antigen, CD8⁺ T cells from animals treated with bacteria transfected with pFAP or the empty vector were purified. These cells were then stimulated with γ-irradiated tumor target cells, and incubated with live tumor target cells that were transiently transfected with either GFP as a control or GFP/pFap. As shown in FIG. 3, Panel B, only CD8⁺ T cells purified from mice treated with pFap were able to induce nuclear apoptosis as assessed by staining of pFap transfected target cells with Hoechst-33342 dye.

Splenocytes of treated mice were also used in a conventional ⁵¹Cr-release assay. To this end, splenocytes from mice treated with Composition 1 and the control vaccine were incubated for 5 days with γ-irradiated A31 fibroblasts, which were retrovirally infected with pFap. Stimulated splenocytes were then incubated for 4 hours with labeled A31-pFap cells and the percentage of lysis was calculated. Splenocytes from mice treated with Composition 1 were able to lyse significantly more fibroblasts than those from empty vector controls at target-to-effector ratios of 1:100 and 1:25 (FIG. 3, Panel C). Co-incubation with anti-MHC class I antibodies eliminated this effect (FIG. 3, Panel D).

In an additional evaluation, mice were treated three times with either Composition 1 or the control vaccine and then challenged with about 3×10⁴ CT26 tumor cells to investigate CD8⁺ T-cell infiltration in tumors of pFap treated mice. After three weeks, tumors were harvested and single cell suspensions stained for CD3⁺ and CD8⁺ cells and analyzed by FACS. Mice treated with Composition 1 showed a marked increase in CD3⁺CD8⁺ cells in the tumor tissue when compared to the empty vector control (FIG. 3, Panel E).

Tumor sections were also stained with anti-CD8 FITC and DAPI nuclear stain. Using confocal microscopy, tumors of mice treated with Composition 1 showed a more marked infiltration with CD8⁺ cells than mice treated with the control vaccine of Example 1 (FIG. 3. Panel F). Taken together, these findings demonstrate that a DNA composition of the invention, targeting FAP, can overcome peripheral T cell tolerance against the FAP self-antigen.

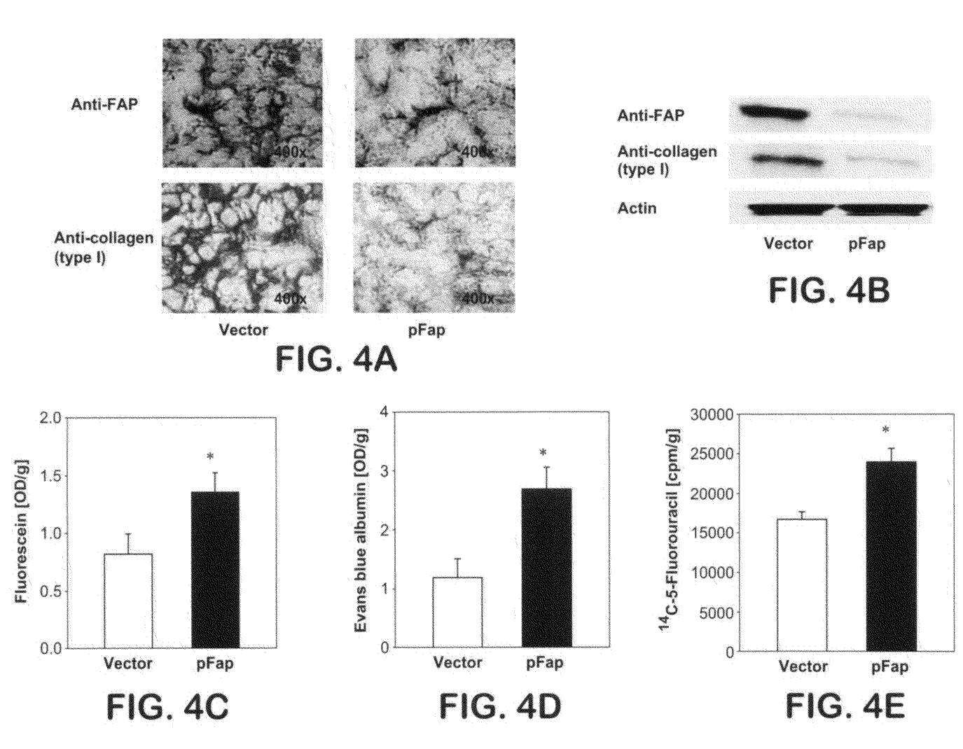

Suppression of collagen type I expression increases intratumor dye uptake. Fibroblasts are the primary source of collagen type I and it has been reported that expression of this molecule correlates inversely with intratumoral uptake of compounds of varying molecular weights. To evaluate whether this same mechanism is applicable to the DNA compositions of the invention, tumor sections of treated mice were stained with antibodies against FAP (FIG. 4, Panel A, upper photos) or against collagen type I (FIG. 4, Panel A, lower photos). A decrease in the expression of FAP as well as collagen type I was detected in groups of mice treated with Composition 1 as compared to mice treated only with the control vaccine (Example 1). Corresponding western blots of these tumor extracts, stained with the same antibodies, revealed about 82.63+/−2.54% decrease in FAP expression (FIG. 4, Panel B, upper photo) and about 76.36+/−2.01% decrease in collagen type I expression (FIG. 4, Panel B, lower photo).

Mice were then injected with three compounds differing in size and structure, i.e. fluorescein (376 Da) (FIG. 4, Panel C), Evans blue albumin (68,500 Da) (FIG. 4, Panel D) or the chemotherapeutic agent 5-fluorouracil (130 Da) (FIG. 4, Panel E) as described above. Tumors of mice treated with pFap DNA Composition 1 incorporated significantly more (p<0.05) of these respective molecules than those of mice administered with the control vaccine.

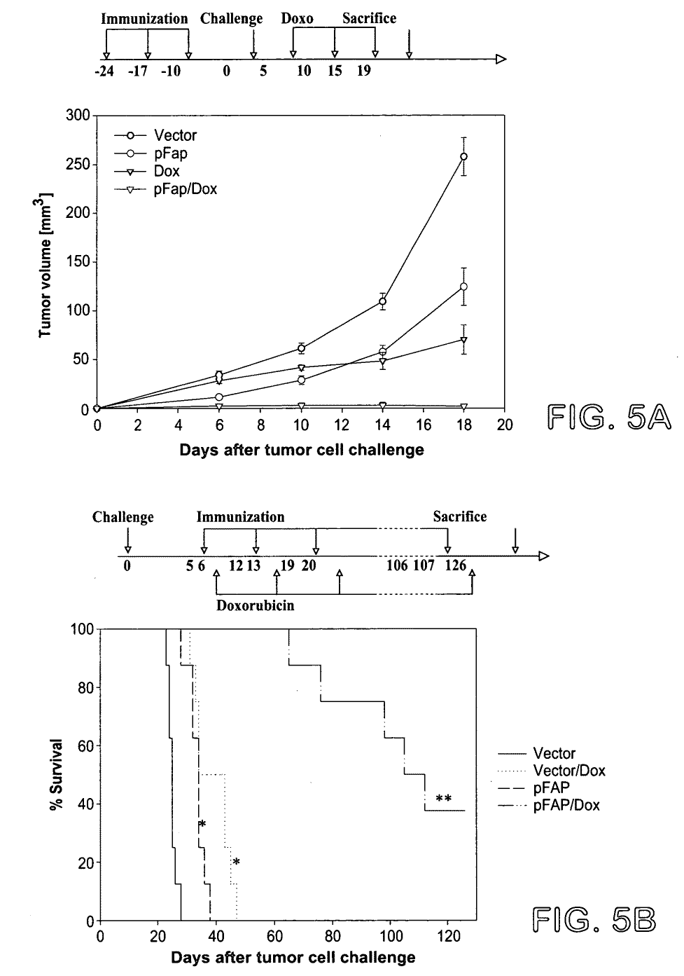

Combination of DNA composition and chemotherapy leads to tumor-rejection. A therapeutic protocol was developed combining treatment with a DNA composition of the invention with the chemotherapeutic drug doxorubicin, to which the D2F2 cells were partially sensitive (FIG. 1, Panel C). BALB/c mice (n=8) were treated with the control vaccine of Example 1, or Composition 1 of Example 1. The treated mice were then challenged orthotopically with D2F2 breast carcinoma cells. These two groups of mice were then treated with either doxorubicin or PBS as a control 5, 10 and 15 days after tumor challenge. As shown in FIG. 5, Panel A, single treatments with either immunotherapy (Composition 1) or chemotherapy were only able to suppress, but not eradicate tumor growth. In contrast, the group of mice treated with Composition 1 and doxorubicin in combination not only revealed a marked inhibition of tumor growth, but also a complete tumor-rejection in 4 of 8 mice (FIG. 5, Panel A).

In another combination therapy approach in a therapeutic setting, BALB/c mice (n=8) were challenged i.v. with D2F2 tumor cells. Once metastasis had been established after 5 days, the mice were treated weekly with Composition 1. One day after each treatment the mice were injected i.v. with doxorubicin. As a result of this combination treatment the life span of these mice exceeded more than 3 times that of control mice that were treated with doxorubicin or Composition 1 alone, and died already after about 35-45 days (FIG. 5, Panel B).

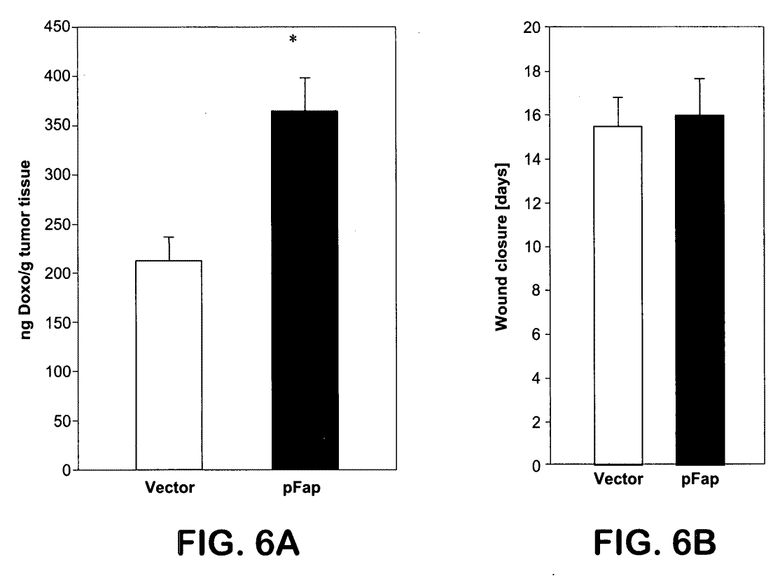

A FAP DNA composition of the invention increases intratumoral uptake of doxorubicin. To determine the doxorubicin concentration in the tumor tissue, mice (n=4) were treated three times at 1-week intervals with Composition 1, challenged 7 days later with 5×10⁵ D2F2 tumor cells, and injected 16 days later i.v. with doxorubicin. The intratumoral drug concentration was determined with liquid chromatography/mass spectrometry (LC-MS). Tissues of mice treated with Composition 1 showed a significant increase of doxorubicin uptake in the tumor (FIG. 6, Panel A) compared to controls. These results are in line with the observed chemical infiltration of tumors in treated mice with fluorescein, albumin, and 5-fluorouracil (FIG. 4, Panels C-D).

The DNA vaccine against FAP does not impair wound healing or damage normal tissue. Since FAP is overexpressed during wound healing, the effects of the DNA compositions of the invention on wound healing were investigated. A circular wound of about 3 mm in diameter was made on the backs of treated BALB/c mice (n=4). Surprisingly, no significant difference in wound healing between treated and non-treated mice was observed (FIG. 6, Panel B). Histological assessment by a mouse pathologist of these wounds after different time points revealed no qualitative abnormalities in the wound healing process. To exclude any autoimmune reactions induced by the DNA compositions of the invention, comprehensive histology of the following tissues and organs was performed: skin, brain, spinal cord, muscle, bone, synovium, heart, aorta, pulmonary artery, thymus, spleen, lymph nodes, bone marrow, parathyroid, adrenal gland, kidney, uterus, vagina, clitoral gland, tongue, liver, lungs, pancreas, stomach, small intestine and colon. No discernable differences were observed compared to control mice.

Numerous variations and modifications of the embodiments described above can be effected without departing from the spirit and scope of the novel features of the invention. No limitations with respect to the specific embodiments illustrated herein are intended or should be inferred.

We claim:

1. A DNA composition effective for eliciting an immune response against tumor stromal cells that express fibroblast activation protein (FAP) comprising a DNA construct that encodes for at least one epitope of FAP, which is expressible in immune cells, and is incorporated in a pharmaceutically acceptable carrier.

2. The DNA composition of claim 1 wherein the DNA construct encodes for human FAP (SEQ ID NO: 2) or a protein that has at least 80% sequence similarity therewith and which includes at least one epitope of human FAP.

3. The DNA composition of claim 1 wherein the DNA construct encodes for human FAP (SEQ ID NO: 2).

4. The DNA composition of claim 1 wherein the DNA construct is a naked DNA construct.

5. The DNA composition of claim 4 wherein the naked DNA construct is in the form of a plasmid.

6. The DNA composition of claim 1 wherein the DNA construct is incorporated in an attenuated viral vector.

7. The DNA composition of claim 1 wherein the DNA construct is incorporated in an attenuated bacterial vector.

8. The DNA composition of claim 7 wherein the attenuated bacterial vector is an attenuated Salmonella typhimurium.

9. The DNA composition of claim 1 wherein the DNA construct is a substantially purified DNA having the polynucleotide sequence consisting of SEQ ID NO: 1 or a polynucleotide sequence that has at least about 80% sequence similarity therewith.

10. The DNA composition of claim 9 wherein the DNA construct is incorporated in an attenuated Salmonella typhimurium vector.

11. The DNA composition of claim 1 wherein the composition further comprises a DNA construct encoding an immune effector protein expressible in immune cells.

12. The DNA composition of claim 11 wherein the immune effector protein is a cytokine.

13. The DNA composition of claim 12 wherein the cytokine is CCL21, IL-2, or CD40LT.

14. A method of inhibiting tumor growth or tumor metastases in a mammal comprising administering to the mammal a DNA composition comprising a DNA construct that encodes for at least one epitope of fibroblast activation protein (FAP) that is expressible in immune cells of the mammal, and is incorporated in a pharmaceutically acceptable carrier; the composition being administered in an amount sufficient to elicit an immune response against cells in the mammal that express the FAP antigen.

15. The method of claim 14 wherein the mammal is a human.

16. The method of claim 14 wherein the DNA construct is incorporated in an attenuated bacterial vector.

17. The method of claim 16 wherein the attenuated bacterial vector is an attenuated Salmonella typhimurium.

18. The method of claim 14 wherein the composition is administered orally.

19. A method of inhibiting tumor growth or tumor metastases in a mammal comprising the steps of administering to the mammal a DNA composition comprising a DNA construct that encodes for at least one epitope of fibroblast activation protein (FAP) that is expressible in immune cells of the mammal, and is incorporated in a pharmaceutically acceptable carrier; and subsequently administering to the mammal an antitumor effective amount of an antitumor chemotherapeutic agent; the composition being administered in an amount sufficient to elicit an immune response against cells in the mammal that express the FAP antigen.

20. The method of claim 19 wherein the mammal is a human.

21. The method of claim 19 wherein the chemotherapeutic agent is doxorubicin.

22. The method of claim 19 wherein the DNA construct is incorporated in an attenuated bacterial vector.

23. The method of claim 22 wherein the attenuated bacterial vector is an attenuated Salmonella typhimurium.

24. The method of claim 19 wherein the composition is administered orally.

25. A method of enhancing the uptake of a chemotherapeutic agent in a mammal comprising the steps of administering to the mammal a DNA composition comprising a DNA construct that encodes for at least one epitope of fibroblast activation protein (FAP) that is expressible in immune cells of the mammal, and is incorporated in a pharmaceutically acceptable carrier, and subsequently administering to the mammal an effective amount of a chemotherapeutic agent; the composition being administered in an amount sufficient to elicit an immune response against cells in the mammal that express the FAP antigen.

26. The method of claim 25 wherein the mammal is a human.

27. The method of claim 25 wherein the chemotherapeutic agent is doxorubicin.

28. The method of claim 25 wherein the DNA construct is incorporated in an attenuated bacterial vector.

29. The method of claim 28 wherein the attenuated bacterial vector is an attenuated Salmonella typhimurium.

30. The method of claim 25 wherein the composition is administered orally.

31. A plasmid vector comprising a DNA construct that encodes for at least one epitope of human fibroblast activation protein (FAP).

32. The vector of claim 31 wherein the DNA construct encodes for human FAP (SEQ ID NO: 2) or a protein that has at least 80% sequence similarity therewith and which includes at least one epitope of human FAP.

33. The vector of claim 31 further comprising a DNA construct encoding an immune effector protein.

34. The vector of claim 33 wherein the immune effector protein is a cytokine.

35. The vector of claim 34 wherein the cytokine is CCL21, IL-2, or CD40LT.


SEQUENCE LISTING (16 sequences)

SEQ ID NO: 1 - DNA, 2648 bp, homo sapiens
ctgaagacagaattagctaactttcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaagtgcgaaaaaaaaaaaaaaaaaaaaaaaaaaaaaa

SEQ ID NO: 2 - protein, 760 aa, homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 3 - DNA, 2615 bp, mus musculus
ccacgcgtccgcacagatgcggtgacccacgctgtgcagtgagaatcagctaacttgcaaaaacatctggaaaaatgaagacatggctgaaaactgtctttggagttaccaccctggctgcgcttgctttagtggtgatatgcattgtcttacgtccctcaagagtttacaaacctgaaggaaacacaaagagagctcttaccttgaaggatattttaaatggaacattctcatataaaacatattttcccaactggatttcagaacaagaatatcttcatcaatctgaggatgataacatagtattttataatattgaaacaagagaatcatatatcattttgagtaatagcaccatgaaaagtgtgaatgctacagattatggtttgtcacctgatcggcaatttgtgtatctagaaagtgattattcaaagctctggcgatattcatacacagcgacatactacatctacgaccttcagaatggggaatttgtaagaggatacgagctccctcgtccaattcagtatctatgctggtcgcctgttgggagtaaattagcatatgtatatcaaaacaatatttatttgaaacaaagaccaggagatccaccttttcaaataacttatactggaagagaaaatagaatatttaatggaataccagactgggtttatgaagaggaaatgcttgccacaaaatatgctctttggtggtctccagatggaaaatttttggcatatgtagaatttaatgattcagatataccaattattgcctattcttattatggtgatggacagtatcctagaactataaatattccatatccaaaggctggggctaagaatccggttgttcgtgtttttattgttgacaccacctaccctcaccacgtgggcccaatggaagtgccagttccagaaatgatagcctcaagtgactattatttcagctggctcacatgggtgtccagtgaacgagtatgcttgcagtggctaaaaagagtgcagaatgtctcagtcctgtctatatgtgatttcagggaagactggcatgcatgggaatgtccaaagaaccaggagcatgtagaagaaagcagaacaggatgggctggtggattctttgtttcgacaccagcttttagccaggatgccacttcttactacaaaatatttagcgacaaggatggttacaaacatattcactacatcaaagacactgtggaaaatgctattcaaattacaagtggcaagtgggaggccatatatatattccgcgtaacacaggattcactgttttattctagcaatgaatttgaaggttaccctggaagaagaaacatctacagaattagcattggaaactctcctccgagcaagaagtgtgttacttgccatctaaggaaagaaaggtgccaatattacacagcaagtttcagctacaaagccaagtactatgcactcgtctgctatggccctggcctccccatttccaccctccatgatggccgcacagaccaagaaatacaagtattagaagaaaacaaagaactggaaaattctctgagaaatatccagctgcctaaagtggagattaagaagctcaaagacgggggactgactttctggtacaagatgattctgcctcctcagtttgacagatcaaagaagtaccctttgctaattcaagtgtatggtggtccttgtagccagagtgttaagtctgtgtttgctgttaattggataacttatctcgcaagtaaggaggggatagtcattgccctggtagatggtcggggcactgctttccaaggtgacaaattcctgcatgccgtgtatcgaaaactgggtgtatatgaagttgaggaccagctcacagctgtcagaaaattcatagaaatgggtttcattgatgaagaaagaatagccatatggggctggtcctacggaggttatgtttcatccctggcccttgcatctggaactggtcttttcaaatgtggcatagcagtggctccagtctccagctgggaatattacgcatctatctactcagagagattcatgggcctcccaacaaaggacgacaatctcgaacactataaaaattcaactgtgatggcaagagcagaatatttcagaaatgtagactatcttctcatccacggaacagcagatgataatgtgcactttcagaactcagcacagattgctaaagctttggttaatgcacaagtggatttccaggcgatgtggtactctgaccagaaccatggtatattatctgggcgctcccagaatcatttatatacccacatgacgcacttcctcaagcaatgcttttctttatcagactgaaccaatgcaagtactagcgtgtaggacagtctgaaggcttcacatggacgacttggaccatgaatattgtaaaagccgctaaaaatttaggagggctcaggaggttaggctcggtattgtttacatttccctatgctctgtgaaagaagagaaaaggggagccacacatcttgctttggacacaatgttttatcacctgttcattgccgggtagcggaaataaagtcagatgctcaggttaaaaaaaaaaaaaaa

SEQ ID NO: 4 - protein, 761 aa, mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSD

SEQ ID NO: 5 - DNA, 814 bp, homo sapiens
atcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacgtatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 6 - protein, 153 aa, homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - DNA, 939 bp, mus musculus
atcacccttgctaatcactcctcacagtgacctcaagtcctgcaggcatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataactatgtacctcctgcttacaacacataaggctctctatttatttaaatatttaactttaatttatttttggatgtattgtttactatcttttgtaactactagtcttcagatgataaatatggatctttaaagattctttttgtaagccccaagggctcaaaaatgttttaaactatttatctgaaattatttattatattgaattgttaaatatcatgtgtaggtagactcattaataaaagtatttagatgattcaaatataaataagctcagatgtctgtcatttttaggacagcacaaagtaagcgctaaaataacttctcagttattcctgtgaactctatgttaatcagtgttttcaagaaataaagctctcctct

SEQ ID NO: 8 - protein, 160 aa, mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFC

SEQ ID NO: 9 - DNA, 852 bp, homo sapiens
cttgcagctgcccacctcaccctcagctctggcctcttactcaccctctaccacagacatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatagcccagtgagcagcctggagccctggagaccccaccagcctcaccaacgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccgc

SEQ ID NO: 10 - protein, 134 aa, homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 11 - DNA, 615 bp, mus musculus
gaattcggccaaagaggcctacggccaaagagggctaaacttgcggctgtccatctcacctacagctctggtctcatcctcaactcaaccacaatcatggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagaggatagcccagtagcccgcctggagcccaggagatcccccacgaacttcaagctgggtggttcacggtccaactcacaggcaaagagggagctagaaaacagactcaggagccgctagtcgag

SEQ ID NO: 12 - protein, 133 aa, mus musculus
MAQMMTLSLLSLVLALCIPWTQGSDGGGQDCCLKYSQKKIPYSIVRGYRKQEPSLGCPIPAILFSPRKHSKPELCANPEEGWVQNLMRRLDQPPAPGKQSPGCRKNRGTSKSGKKGKGSKGCKRTEQTQPSRG

SEQ ID NO: 13 - DNA, 878 bp, mus musculus
atcccagcccacgcacagacccccaacttgcagctgcccacctcaccctcagctctggcctcttactcaccctctaccacagacatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatagcccagtgagcagcctggagccctggagaccccaccagcctcaccagcgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccaa

SEQ ID NO: 14 - protein, 134 aa, mus musculus; identical to SEQ ID NO: 10

SEQ ID NO: 15 - DNA, 1816 bp, homo sapiens
cttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaacccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaaaa

SEQ ID NO: 16 - protein, 261 aa, homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL